(19) United States
(12) Patent Application Publication
(10) Pub. No.: US 2010/0286254 A1
(43) Pub. Date: Nov. 11, 2010

(54) CRYSTALLINE FORMS OF DMXAA SODIUM SALT

(75) Inventors: Fritz Blatter, Reinach (CH); Rolf Hilfiker, Allschwil (CH)

Correspondence Address: LAHIVE & COCKFIELD, LLP;FLOOR 30, SUITE 3000, ONE POST OFFICE SQUARE, BOSTON, MA, 02109, US

(73) Assignee: ANTISOMA RESEARCH LIMITED, London (GB)

(21) Appl. No.: 12/680,925

(22) PCT Filed: Oct. 21, 2008
(86) PCT No.: PCT/GB2008/003558

§ 371 Date: Jul. 12, 2010

Related U.S. Application Data
(60) Provisional application No. 60/981,929, filed on Oct. 23, 2007.

(51) Int. Cl. A61K 31/352; C07D 311/82; A61P 35/00

(52) U.S. Cl. 514/455; 549/392

(57) ABSTRACT

The present invention relates to pharmaceutically stable crystalline forms of (5,6-Dimethyl-9-oxo-9H-xanthene-4-yl)acetic acid (DMXAA) sodium salt, processes for preparing those stable crystalline forms; pharmaceutical compositions comprising at least one of those crystalline forms in solid form or in dissolved form and a pharmaceutically acceptable carrier. Disclosed are methods of using those pharmaceutical compositions to treat tumours, optionally in combination with other active pharmaceutical agents.

### CROSS-REFERENCE TO RELATED APPLICATIONS

This application claims benefit under 35 U.S.C. §119(e) to a U.S. Provisional Application Ser. No. 60/981,929, filed Oct. 23, 2007, which is incorporated herein by reference in its entirety.

### FIELD OF THE INVENTION

The present invention relates to pharmaceutically stable crystalline forms of 5,6-dimethyl-oxo-xanthene-4-acetic acid (DMXAA) sodium salt, processes for preparing those stable crystalline forms, pharmaceutical compositions comprising at least one of those crystalline forms in solid form or in dissolved form and a pharmaceutically acceptable carrier, and methods of using those pharmaceutical compositions to treat tumours, optionally in combination with other active pharmaceutical agents.

### BACKGROUND TO THE INVENTION

(5,6-Dimethyl-1-oxo-9H-xanthene-4-yl) acetic acid (DMXAA) of the following formula

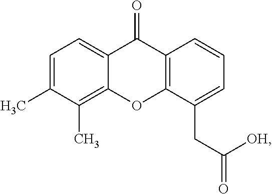

was first disclosed in European Patent EP 0 278 176 as compound 34. It is an anti-tumour agent with a number of activities, including notably the ability to shut down blood flow in tumours (B. G. Slim at al., 2000, Cancer Res. 60, 4582-4588 et R. Murata et al., 2001, Int. Radiat. Biol. 77, 195-204), induce production of tumour necrosis factor (L. M. Ching et al., 1999, Cancer Res. 59, 3304-3307 et W. R. Joseph et al., 1699, Cancer Res. 59, 633-638), and inhibit tumour angiogenesis (Z. Cao et al., 2001, Cancer Res. 61, 1517-1521).

Three phase I Clinical trials of DMXAA as a monotherapy have recently been completed, with dynamic MRI (Magnetic Resonance Imaging) showing that it induces a significant reduction in tumour blood flow at well-tolerated doses. DMXAA is thus one of the first vascular disrupting agents (VDAs) for which activity (irreversible inhibition of tumour blood flow) has been well documented in human tumours. These findings are in agreement with preclinical studies using syngeneic murine tumours or human tumour xenografts, which showed that its antivascular activity produced prolonged inhibition of tumour blood flow leading to extensive regions of haemorrhagic necrosis.

However, despite these observations, few tumour responses were achieved in these phase I trials, indicating that DMXAA may have limited potential as a monotherapy for the treatment of cancer.

Although not showing much promise as a monotherapy, DMXAA has also been considered for use in the treatment, of cancer in combination with another form of therapy, such as radiotherapy, hyperthermia, or photodynamic therapy, or in combination with another chemotherapeutic agent (see “Flavones and xanthenones as vascular-disrupting agents”, Slim, Brown G et al., in “Vascular-Targeted Therapies in Oncology”, 2006, Ed. Siemann, Dietmar W., John Wiley & Sons Ltd, Chichester UK).

To this extent, various active agents have been disclosed for co-administration with DMXAA with a view to treating cancer. These active agents include taxanes (paclitaxel and docetaxel), platins (cisplatin and carboplatin), cyclophosphamide, vinca alkaloids (vincristine, vinblastine), antimetabolites (gemcitabine), topoisomerase II inhibitors (etoposide) and anthracyclines (doxorubicin), tumour necrosis factor (TNF) stimulating compounds and immunomodulatory compounds such as intracellular adhesion molecules, (ICAMs) or thalidomide, non steroidal anti-inflammatory drugs (NSAIDs) such as diclofenac, EGFR signalling pathway inhibitors (e.g. a monoclonal antibody such as cetuximab, or a tyrosine kinase inhibitor such as erlotinib or gefitinib) and VEGF binders (such as bevacizumab). See e.g. WO 02/09700, WO 03/020259; WO 03/080044, WO 2007/023307 and U.S. Pat. No. 6,667,337.

DMXAA is generally administered intravenously in a formulation comprising a pharmaceutically acceptable salt of DMXAA dissolved in an aqueous solvent at a physiologically acceptable pH. Other modes of administration, in particular oral, rectal, vaginal, ophthalmic, nasal, topical, parenteral, transdermal and intracranial have also been described, notably in WO 2005/027974 and WO 2004/039363.

An example of a pharmaceutically acceptable salt is DMXAA sodium salt.

The synthesis of DMXAA sodium salt is reported by G. W, Rewcastle et al., 1991, J. Med. Chem. 34, 217-222: it is a eight-step process giving in the 7^{th} step DMXAA and in the 8^{th} step, as intermediate an amorphous form of DMXAA sodium salt. Although the amorphous material is described in G. W, Rewcastle et al. as being recrystallised (from a mixture of methanol and ethyl acetate), our own reproduction of the preparative method indicates that the product obtained was most likely a crystalline methanol solvate of DMXAA sodium salt, the solvate containing in the region of 13-20% by weight of methanol.

Methanol not being a physiologically, acceptable solvent; a crystalline methanol solvate of DMXAA sodium salt cannot be used in a pharmaceutical composition or for preparing a pharmaceutical composition. Indeed, the International Conference on Harmonisation of Technical Requirements for Registration of Pharmaceuticals for Human Use classifies methanol as a class 2 solvent that should be limited to a maximum of 30 mg/day (ICH guideline Q3C, 17 Jul. 1997), and to 3000 ppm in any pharmaceutical product. Using a crystalline methanol solvate of DMXAA sodium for preparing a pharmaceutical solution makes it difficult to attain the required low level of methanol.

There are various problems associated with using amorphous DMXAA sodium salt in the preparation of pharmaceutical compositions of DMXAA. For example, the amorphous salt is very hygroscopic. This not only makes it difficult to handle (sticky), but also means that it is difficult to measure precise quantities of the active moiety. This is because the quantity of water present in different samples can vary widely. Further, depending upon the atmospheric conditions that it is exposed to, the quantity of water present in a single sample can vary considerably over time. The difficulty of controlling the water content of the amorphous form thus makes it difficult to obtain the required uniformity in the preparation of set dosages of DMXAA sodium salt.

The invention concerns a crystalline DMXAA sodium salt in the form of an anhydrate or a solvate. In particular, the invention concerns a crystalline DMXAA sodium salt in the form of an anhydrate or a solvate with a physiologically acceptable solvent.

### SUMMARY OF THE INVENTION

Disclosed herein are compositions and methods of a crystalline DMXAA sodium salt in the form of an anhydrate or a solvate.

In one aspect, there is provided a crystalline DMXAA sodium salt in the form of an anhydrate or a solvate.

In another aspect, there is provided a pharmaceutical composition containing a crystalline DMXAA sodium salt of the invention and a pharmaceutically acceptable carrier or diluent.

In another aspect, there is provided a process of preparing a crystalline DMXAA sodium salt of the invention.

In another aspect, there is provided a method of preparing a crystalline DMXAA sodium salt of the invention.

In yet another aspect, there is provided a method of treating cancer in a patient in need of such treatment comprising administering an effective amount of a crystalline DMXAA sodium salt of the invention to the patient.

In another aspect, there is provided a use of the crystalline DMXAA sodium salt of the invention for the preparation of a medicament for the treatment of cancer such as a solid tumour.

In yet another aspect, there is provided a kit of parts comprising the crystalline DMXAA sodium salt of the invention.

In each of these aspects, the crystalline DMXAA sodium salt is any one or more of form A, B, C, D, E, F, G, H, I, J, K, L, or M.

### BRIEF DESCRIPTION OF THE DRAWINGS

FIG. 1 shows a characteristic X-ray powder diffraction pattern of crystalline fan

FIG. 2 shows a characteristic X-ray powder diffraction pattern of form A.

FIG. 3 shows a characteristic X-ray Powder diffraction pattern of form B

FIG. 4 shows a characteristic X-ray powder diffraction pattern of form C.

FIG. 5 shows a characteristic X-ray powder diffraction pattern of form D.

FIG. 6 shows a characteristic X-ray powder diffraction pattern of form E.

FIG. 7 shows a characteristic X-ray powder diffraction pattern of form F.

FIG. 8 shows a characteristic X-ray powder diffraction pattern of form G.

FIG. 9 shows a characteristic X-ray powder diffraction pattern of form H.

FIG. 10 shows a characteristic X-ray powder diffraction pattern of form I.

FIG. 11 shows a characteristic X-ray powder diffraction pattern of form J.

FIG. 12 shows a characteristic X-ray powder diffraction pattern of form K. The upper trace shown between 2 theta values of 10 and 40 degrees represents a 10-fold magnification of the lower trace.

FIG. 13 shows a characteristic X-ray powder diffraction pattern form L.

FIG. 14 shows a characteristic Raman spectrum of form B.

FIG. 16 shows a characteristic Raman spectrum of form C.

FIG. 16 shows a characteristic Raman spectrum of form F.

FIG. 17 shows the Dynamic Vapour Sorption diagram of forms B, C and E.

### DETAILED DESCRIPTION. OF THE INVENTION

Throughout this disclosure, various publications, patents and published patent specifications are referenced by an identifying citation. The disclosures of these publications, patents and published patent specifications are hereby incorporated by reference into the present disclosure to more fully describe the state of the art to which this invention pertains.

The terms “comprising”, “treat”, “treatment”, “treating”; “an effective amount”, “physiologically acceptable pH”, “solvate”, “patient”, “amorphous form” and “crystalline form” have meanings that will be well understood by those skilled in the art. However, for the avoidance of doubt, embodiments of the present invention include those in which these term take the meanings identified below.

As used in the specification and claims, the singular form “a,” “an” and “the” include singular and plural references unless the context clearly dictates otherwise. For example, “a crystalline form” includes a single crystal as well as a plurality of crystals, including mixtures thereof unless otherwise noted.

As used herein, the term “comprising” is intended to mean that the compositions and methods include the recited elements, but not excluding others. “Consisting essentially of” when used to define compositions and methods, shall mean excluding other elements of any essential significance to the composition or method, “Consisting of” shall mean excluding more than trace elements of other ingredients for claimed compositions and substantial method steps. Embodiments defined by each of these transition terms are within the scope of this invention. Accordingly, it is intended that the methods and compositions can include additional steps and components (comprising) or alternatively including steps and compositions of no significance (consisting essentially of) or alternatively, intending only the stated method steps or compositions (consisting of).

All numerical designations, e.g., pH, temperature, time, concentration, and molecular weight including ranges, are approximations which are varied (+) or (−) by increments of 0.1. It is to be understood, although not always explicitly stated that all numerical designations are preceded by the term “about”. The term “about” also includes the exact value “X” in addition to minor increments of “X” such as “X+0.1” or “X−0.1.” It also is to be understood, although not always explicitly stated, that the reagents described herein are merely exemplary and that equivalents of such are known in the art.

The term “treating” as used herein is intended, to encompass curing as well as ameliorating at least one symptom of the condition or disease. For example, in the case of cancer or solid tumours, a response to treatment includes a reduction in cachexia, increase in survival time, elongation in time to tumour progression, reduction in tumour mass, reduction in tumour burden and/or a prolongation, in time to tumour metastasis, time to tumour recurrence, tumour response, complete response, partial response, stable disease, progressive disease, progression free survival, overall Survival, each as measured by standards set by the National Cancer Institute and the U.S. Food and Drug Administration for the approval of new drugs. See Johnson et al. (2003) J. Clin. Oncol. 21(7):1404-1411.

As use, “solid tumour” intends en abnormal mass of tissue that usually does not contain cysts or liquid areas. Solid tumours May be benign (not cancerous) or malignant (cancerous). Different types of solid tumours are named, for the types of cells that form them Examples of solid tumours are sarcomas, carcinomas, and lymphomas.

A “complete response” (CR) to a therapy defines patients with evaluable but non-measurable disease, whose tumour and all evidence of disease had disappeared.

A “partial response” (PR) to a therapy defines patients with anything less than complete response and simply categorized as demonstrating partial response.

A “stable disease” (SD) indicates that the patient is stable.

A “progressive disease” (PD) indicates that the tumour has grown (i.e. become larger), spread, (i.e. metastasized to another tissue or organ) or the overall cancer has gotten worse following treatment. For example, tumour growth of more than 20 percent since the start of treatment typically indicates progressive disease.

An “overall survival” (OS) intends a prolongation in life expectancy as compared to naïve or untreated individuals or patients.

A “progression free survival” (PFS) or “time to tumour progression” (TTP) indicates the length of time during and after treatment that the cancer or tumour does not grow. Progression-free survival includes the amount of time patients have experienced a complete response or a partial response, as well as the amount of time patients have experienced stable disease.

The term “an effective amount” intends to indicate the amount of a compound or agent administered or delivered to the patient which is most likely to result in the desired response to treatment. The amount is empirically determined by the patient's clinical parameters including, but not limited to the stage of disease, age, gender, histology, and likelihood for tumour recurrence.

The term “physiologically acceptable pH” refers to a pH at a level of about 3.5 to about 8.6.

The terms “solvate” or “solvates” of a crystalline DMXAA sodium salt refer to the crystalline DMXAA sodium salt bound to a stoichiometric or non-stoichiometric amount of a solvent. Solvates include solvates of all forms of the crystalline DMXAA sodium salt. Preferred solvents are volatile, non-toxic, and/or acceptable for administration to patients in trace amounts. Solvates include water.

The term “patient” refers to mammals and includes humans and non-human mammals which include, but are not limited ti simians, murines, rats and leporids.

The term “amorphous form” refers to a compound having no definite crystal structure or form.

The term “crystalline form” refers to a compound which is in a solid form and in which the constituent atoms, molecules, or ions are packed in a regularly ordered, repeating pattern extending in all three spatial dimensions. The physical properties of the various crystalline forms can differ due to the presence of solvates or other molecules incorporated into the lattice of the crystalline form.

The crystalline DMXAA sodium salt provided herein may have, the following significant advantages:
Ease of handling and processing in the manufacture and preparation of pharmaceutical compositions. The obtained particle size distributions in a large scale crystallisation process may be easier to control.
Defined product stoichiometry in terms of solvate molecules (e.g. water), if present. This might allow for an amount of pharmaceutically, active substance to be more accurately measured/dosed.
Improved chemical and/or physical stability (e.g. through having lower free energy than the amorphous form of DMXAA sodium salt)

The crystalline DMXAA sodium salt of the invention is essentially or substantially freely soluble in water. The term “freely soluble in water” here means that the aqueous solubility is at least 100 mg/mL at 22° C. and pH 8.3.

The term “anhydrate” means here a water and solvent free crystalline form of DMXAA sodium salt that may contain up to a few % of surface adsorbed water, such as less than about 3% (e.g. less than about 2.5, 2.0, 1.5, 1.0, 0.5 or 0.25%). Such water adsorbed on the surface of a given crystalline form does generally not affect the crystalline structure, and the powder X-ray diffraction pattern is essentially or substantially unchanged with respect to a completely water-free form.

DMXAA sodium salt in the form of a solvate with a physiologically acceptable solvent may be a crystalline hydrate, a crystalline solvate of an organic solvent, or a crystalline mixed hydrate-solvate of an organic solvent, wherein the organic solvent is a physiologically acceptable solvent.

The physiologically acceptable solvent is a solvent having no or low toxicity when administered at a small dose to the human body. Examples of physiologically acceptable solvents are water, class 3 solvents according to the ICH Q3C guideline mentioned above, which, includes alcohols such as ethanol or isopropanol, esters, and tert-butyl methyl ether. However, solvents with higher molecular weights such as esters of fatty acids, and glycols, e.g. polyethylene glycol may also be acceptable.

The physiologically acceptable solvent is in certain embodiments, selected from the group consisting of water, ethanol and isopropanol. In some embodiments, the physiologically acceptable solvent is water.

When the crystalline DMXAA sodium salt is an anhydrate, it is generally prepared by a process comprising suspending amorphous DMXAA sodium salt or any crystalline form of DMXAA sodium salt in an organic solvent, stirring the obtained suspension until formation of the anhydrate crystalline form is complete, filtering and drying under suitable conditions, e.g. in dry air at ambient temperature. A crystalline DMXAA sodium salt anhydrate may also be prepared by evaporating water under mild conditions from a crystalline DMXAA sodium salt hydrate, A further process to produce crystalline anhydrate forms comprises dissolving DMXAA in a suitable organic solvent (e.g. at above ambient temperature), thereafter adding one equivalent of sodium in water-free form, for instance in form of sodium ethanolate. This step is followed by cooling the obtained mixture, seeding with the desired crystalline form, and if necessary, stirring the obtained suspension at the final temperature until a completely crystalline product is obtained, and isolating the obtained crystalline material by filtration. Suitable organic solvents that may be mentioned are ICH Q3C solvents in which DMXAA is reasonably soluble and DMXAA sodium salt does not form a solvated form. Particular solvents that may be mentioned are, for instance, methyl ethyl ketone, THF or ethyl acetate.

In addition to advantages described above, anhydrate forms of crystalline DMXAA sodium salt may have one or both of the following significant advantages:
such forms can be produced essentially free of residual solvent; and/or
simple drying protocols can be applied.

When the crystalline DMXAA sodium salt is a hydrate, it is generally prepared by a process comprising stirring of a suspension of an amorphous form or any crystalline form of DMXAA sodium salt in an organic solvent containing water as a co-solvent in a ratio that results in a suitable water activity, filtering off the obtained crystalline form and drying it under suitable conditions. Organic solvents that may be mentioned in this respect include acetone. A “suitable water activity” here means a water activity at which the hydrate, is thermodynamically stable. A crystalline DMXAA sodium salt hydrate may also be prepared by suspending an amorphous form or any crystalline form of DMXAA sodium salt in an appropriate aqueous solvent, stirring, filtering off the obtained crystalline form and drying it under suitable conditions.

A further process to produce crystalline hydrate forms comprises dissolving DMXAA in a suitable organic solvent (e.g. at above ambient temperature), thereafter adding one equivalent of sodium, for instance in form of an aqueous NaOH solution in a ratio and concentration that results into a suitable water activity in the resulting solvent-water mixture to obtain the desired hydrate form.

This step is followed by cooling the obtained mixture, seeding with the desired crystalline form, and if necessary, stirring the obtained suspension at the final temperature until a completely crystalline product is obtained, and isolating the obtained crystalline material by filtration. Example of suitable organic solvent includes, but is not limited to, ICH Q3C solvent in which DMXAA is reasonably or substantially soluble and DMXAA sodium salt does not form a solvated form. Other examples of solvents are, for instance, methyl ethyl ketone, THF or ethyl acetate, which exhibit sufficient miscibility with water.

In addition to advantages described above, hydrate forms of crystalline DMXAA sodium salt may have on or more of the following significant advantages:
hydrate forms are not hygroscopic and require less moisture protection measures;
large scale processes to produce hydrate forms that are essentially free of undesired organic solvents are more straightforward to develop and therefore more easily accessible; and/or
high stability under moisture conditions (for instance at 40° C./75% r.h.).

When, the crystalline DMXAA sodium salt FS solvate of a physiologically acceptable organic solvent, it is generally prepared by a process comprising suspending an amorphous form or any crystalline form of DMXAA sodium salt in a physiologically acceptable organic solvent, stirring, filtering off the obtained crystalline form and drying it under suitable conditions. The ethanol solvate can also be produced by dissolving DMXAA in ethanol above ambient temperature, (e.g. at about 65° C.), adding one equivalent of sodium in the form of sodium ethanolate, cooling the obtained mixture to 20° C. and then isolating the resulting, DMXAA sodium salt ethanol solvate by filtration.

When the crystalline DMXAA sodium salt is a mixed hydrate-solvate of a physiologically acceptable organic solvent, it is generally prepared by a process comprising dissolving an amorphous form or any crystalline form of DMXAA sodium salt in water, adding a physiologically acceptable organic solvent, stirring, filtering off the obtained crystalline form and drying it under suitable conditions.

The crystalline DMXAA sodium salt of the invention can be used in medical therapy, in particular for treating malignancy of any type including, for example, cancer of the lung, breast, testes, prostate, gut including colon, ovary, skin, kidney, pancreas, and lymphatic organs, cervix, liver, brain and leukaemias.

This invention concerns a pharmaceutical composition comprising:
(a) as active ingredient, a crystalline DMXAA sodium salt in the form of an anhydrate or a solvate; and
(b) a pharmaceutically acceptable carrier or diluent.

The invention thus also concerns a pharmaceutical composition containing:
(a) as active ingredient, a crystalline DMXAA sodium salt in the form of an anhydrate or a solvate with a physiologically acceptable solvent; and
(b) a pharmaceutically acceptable carrier or diluent.

In that pharmaceutical composition, the crystalline DMXAA sodium salt is present either in solid form, in particular in formulations for oral administration such as tablets, pills or capsules, or dissolved in an aqueous solvent at a physiologically acceptable pH, e.g. in formulations for intravenous injection.

Pharmaceutical compositions containing as active substance the crystalline DMXAA sodium salt in solid form may have notably one or both of the following significant advantages (over pharmaceutical compositions containing an amorphous form of DMXAA sodium salt in solid state):
greater stability (as a crystalline DMXAA sodium salt is more stable than DMXAA sodium salt in an amorphous form); and/or
greater uniformity in dosage levels (as the amount of active substance can be dosed with a much better reliability compared to an amorphous form of DMXAA sodium salt, which is sticky and has a water content that is difficult to control/accurately quantify).

Pharmaceutical compositions containing as active substance dissolved crystalline DMXAA sodium salt may have one or both of the significant advantages:
compared to compositions prepared from amorphous DMXAA sodium salt, greater uniformity in dosage levels (for much the same reasons as discussed above); and/or
compared to compositions prepared from the methanol solvate of DMXAA sodium salt, elimination from the final composition of a solvent (methanol) that is not physiologically acceptable.

In some embodiments, there is provided a method of preparing a pharmaceutical composition containing as active ingredient a crystalline DMXAA sodium salt in form of an anhydrate or a solvate, comprising, mixing the crystalline DMXAA sodium salt in an aqueous solution having a physiologically acceptable pH.

A pharmaceutical composition containing as active substance a dissolved crystalline DMXAA sodium salt may be prepared by dissolving crystalline DMXAA sodium salt, generally in powder form, in an aqueous solvent having a physiologically acceptable pH, e.g. a 0.01M tris (tris(hydroxymethyl)aminomethane) buffer solution where the pH has been adjusted to 7.8-8.6 by addition of an acid such as, hydrochloric acid or any other suitable acid known in the art such as sulphuric acid.

The pharmaceutical compositions may be used far treating cancer or solid tumours in combination with another form of therapy such as radiotherapy, hyperthermia, or photodynamic therapy, or simultaneous or sequential administration of one or more further pharmaceutically active compound (e.g. a compound acting in synergy with DMXAA in treating the tumour). The further pharmaceutically active compound may, for example, be selected from one or more of taxanes (e.g. paclitaxel and docetaxel), platins (e.g. cisplatin and carboplatin), cyclophosphamide, vinca alkaloids (e.g. vincristine, vinblastine), antimetabolites (e.g. gemcitabine), topoisomerase II inhibitors (e.g. etoposide) and anthracyclines (e.g. doxorubicin), tumour necrosis factor (TNF) stimulating compounds and immunomodulatory compounds such as intracellular adhesion molecules (ICAMs) or thalidomide, non steroidal anti-inflammatory drugs (NSAIDs) such as diclofenac, EGFR signalling pathway inhibitors (e.g. a monoclonal antibody such as cetuximab, or a tyrosine kinase inhibitor such as erlotinib or gefitinib) and VEGF binders (such as bevacizumab).

In particular, the pharmaceutical compositions may be used for treating cancer or a solid tumour (e.g. non-small cell lung cancer (NSCLC), ovarian cancer or prostate cancer) in combination with the sequential administration of a taxane (e.g. paclitaxel or docetaxel) and/or a platin (e.g. carboplatin). Specifically, the pharmaceutical compositions may be used for
(a) treating ovarian cancer or, particularly, NSCLC in combination with the sequential administration of paclitaxel and carboplatin; or
(b) treating prostate cancer (e.g. in patients having a metastatic hormone refractory prostate cancer) in combination with the sequential administration of docetaxel.

Thus, according to further aspects of the invention, there are provided the following.
(A) A crystalline DMXAA sodium salt in the form of an anhydrate or a solvate with a physiologically acceptable solvent for use as a medicament.
(B) crystalline DMXAA sodium salt in the form of an anhydrate or a solvate with a physiologically acceptable solvent for use in the treatment of cancer such as a solid tumour (e.g. ovarian cancer, prostate cancer or, particularly, NSCLC).
(C) The use of a crystalline DMXAA sodium salt in the form of an anhydrate or a solvate with a physiologically acceptable solvent for the preparation of a medicament for the treatment of cancer such as a solid tumour (e.g. ovarian cancer, prostate cancer or, particularly, NSCLC).
(D) A method of treating cancer such as a solid tumour (e.g. ovarian cancer, prostate cancer or, particularly, NSCLC) in a patient in need of such treatment, the method comprising administration of an effective amount of a crystalline DMXAA sodium salt in the form of an anhydrate or a solvate.
(E) A method of treating cancer (e.g. ovarian cancer, prostate cancer or, particularly, NSCLC) in a patient in need of such treatment, said method comprising administration of an effective amount of a crystalline DMXAA sodium salt in the form of an anhydrate or a solvate with a physiologically acceptable solvent.

In relation to (B) above, the use may be in combination with the simultaneous or sequential administration of a further pharmaceutically active compound selected from one or more of taxanes (e.g. paclitaxel and docetaxel), platins (e.g. cisplatin and carboplatin), cyclophosphamide, vinca alkaloids (e.g. vincristine, vinblastine), antimetabolites (e.g. gemcitabine), topoisomerase II inhibitors (e.g. etoposide) and anthracyclines (e.g. doxorubicin), tumour necrosis factor (TNF), stimulating compounds and immunomodulatory compounds such as intracellular adhesion molecules (ICAMs) or thalidomide, non steroidal anti-inflammatory drugs (NSAIDs) such as diclofenac, EGFR signalling pathway inhibitors (e.g. a monoclonal antibody such as cetuximab, or a tyrosine kinase inhibitor such as erlotinib or gefitinib) and VEGF binders (such as bevacizumab).

Similarly, the medicament mentioned in (C) above may be for simultaneous or sequential administration of such a further pharmaceutically active compound. Further, the method of (D) above may additionally comprise the simultaneous or sequential administration of such a further pharmaceutically active compound.

As mentioned above, particular combinations of active agents may be used to treat particular cancers such as solid tumours. Thus, in connection with (B) above, the use may be:
(I) for the treatment of ovarian cancer (e.g. platinum-sensitive recurrent ovarian cancer) or, particularly, NSCLC, in combination with the sequential administration of paclitaxel and carboplatin; or
(II) for the treatment of prostate cancer (e.g. in patients having a metastatic hormone refractory prostate cancer), in combination with the sequential administration of docetaxel.

In relation to (I) above, the crystalline DMXAA sodium salt in the form of an anhydrate or a solvate with a physiologically acceptable solvent may be for use in the treatment of NSCLC in combination with the sequential administration of paclitaxel and carboplatin, wherein each of those three active agents is administered by intravenous injection.

In some embodiments, there is provided a method of treating non-small cell lung cancer in a patient in need of such treatment, the method comprising administering to the patient an effective amount of a crystalline DMXAA sodium salt in form of an anhydrate or a solvate, in combination with the sequential administration of paclitaxel and carboplatin.

In this embodiment the paclitaxel may be administered over a period of 2 to 6 hours (e.g. 2.5 to 3.5 hours, such as about 3 hours). Further, carboplatin may be administered over a period of 20 to 60 minutes, such as 25 to 35 minutes (e.g. about 30 minutes). Also, an aqueous solution of the crystalline DMXAA sodium salt in the form of an anhydrate or a solvate with a physiologically acceptable solvent may be administered over a period of 10 to 45 minutes, such as 15 to 30 minutes (e.g. about 20 minutes).

In certain embodiments, the active agents are administered in the sequential order: paclitaxel, carboplatin and then DMXAA sodium salt. For example, the active agents may be administered via the same intravenous line, with the line being washed for a few minutes (e.g. from 2 to 5 minutes) between different agents.

The qualities of active agents administered may be as follows:
from 150 to 200 mg/kg (e.g. 165 mg/kg) paclitaxel;
from AUC 5 to AUC 7 (e.g. AUC 6) carboplatin;
from 500 to 5000 mg/m² (e.g. from 800 to 3500 or 2200 mg/m², such as from 1000 to 2000 mg/m² or from 1200 to 1800 mg/m² (for example, 1200 mg/m² or 1800 mg/m²) DMXAA.

The dosage of carboplatin can be calculated using a mathematical formulae, which can be based on a patient's pre-existing renal function or renal function and desired platelet nadir. Renal excretion can be the major route of elimination for carboplatin which is administered via injection. The use of dosing formulae, as compared to empirical dose calculation based on body surface area, can allow compensation for patient variations in pretreatment renal function that can otherwise result in either underdosing (in patients with above average renal function) or overdosing (in patients with impaired renal function).

A simple formula for calculating dosage, based upon a patients glomerular filtration rate (GFR in mL/min) and carboplatin for injection target area under the concentration versus time curve (AUC in mg/mL·min), has been proposed by Calvert. GFR can be measured by ⁵¹Cr-EDTA clearance.

Calvert Formula for carboplatin dosing: Total Dose (mg)=(target AUC)×(GFR+25)

With the Calvert formula, the total dose of carboplatin injection is calculated in mg, not mg/m².

In relation to (II) above, the crystalline DMXAA sodium salt in the form of an anhydrate or a solvate with a physiologically acceptable solvent may be for use in the treatment of prostate cancer in combination with the sequential administration of docetaxel, wherein each active agent is administered by intravenous injection.

In some embodiments, there is provided a method of treating metastatic hormone refractory prostate cancer in a patient in need of such treatment, the method comprising administering to the patient an effective, amount of a crystalline DMXAA sodium salt in form of an anhydrate or a solvate, in combination with the sequential administration of docetaxel.

In this embodiment the docetaxel may be administered over a period of 10 minutes to 5 hours (e.g. 30 minutes to 2 or 3 hours, such as 1 hour). Also, an aqueous solution of the crystalline DMXAA sodium salt in the form of an anhydrate or a solvate with a physiologically acceptable solvent may be administered over a period of 10 to 45 minutes, such as 15 to 30 minutes (e.g. about 20 minutes).

In certain embodiments, the active agents are administered in the sequential order: docetaxel followed by DMXAA sodium salt. For example, the active agents may be administered via the same intravenous line, with the line being washed for a few minutes (e.g. from 2 to 5 minutes) between the two different agents.

The quantities of active agents administered may be as follows:
from 25 to 200 mg/m² (e.g. from 40 to 100 or 125 mg/m², such as 60 or 75 mg/m²) docetaxel;
from 500 to 5000 mg/m² (e.g. from 800 to 3500 or 2200 mg/m², such as from 1000 to 2000 mg/m² or from 1200 to 1800 mg/m² (for example, 1200 mg/m² or 180 mg/m²) DMXAA.

Embodiments of the invention that may be mentioned include equivalent specific embodiments (i.e. equivalents of (I), (II), and the sub-embodiments (I) above) of the use of (C) above and the method of (D) above.

The invention further relates to a kit for performing the method of (D) above, namely a kit-of-parts comprising:
(a) a formulation containing a crystalline DMXAA sodium salt in the form of an anhydrate or a solvate with a physiologically acceptable solvent;
(b) one or more separate formulations comprising one or more further pharmaceutically active compounds selected from taxanes (e.g. paclitaxel and docetaxel), platins e.g. cisplatin and carboplatin), cyclophosphamide, vinca alkaloids (e.g. vincristine vinblastine), antimetabolites (e.g. gemcitabine), topoisomerase II inhibitors (e.g. etoposide) and anthracyclines (e.g. doxorubicin), tumour necrosis factor (TNF) stimulating compounds and immunomodulatory compounds such as intracellular adhesion molecules (ICAMs) or thalidomide, non steroidal anti-inflammatory drugs (NSAIDs) such as diclofenac, EGFR signalling pathway inhibitors (e.g. a monoclonal antibody such as cetuximab, or a tyrosine kinase, inhibitor such as erlotinib or gefitinib) and VEGF binders (such as bevacizumab); and
(c) instructions for use of the formulation containing DMXAA together with said one or more separate formulations.

In some embodiments, there is provided a kit-of-parts comprising:
(a) a formulation containing a crystalline DMXAA sodium salt in form of an anhydrate or a solvate;
(b) one or more separate formulations comprising one or more further pharmaceutically active compounds selected from taxanes, platins, cyclophosphamide, vinca alkaloids, antimetabolites topoisomerase II inhibitors, anthracyclines, tumour necrosis factor (TNF) stimulating compounds, immunomodulatory compounds, non steroidal anti-inflammatory drugs (NSAIDs), EGFR signalling pathway inhibitors and VEGF binders; and
(c) instructions for use of the formulation containing DMXAA together with said one or more separate formulations.

In some embodiments, there is provided a pharmaceutical formulation comprising, or alternatively consisting, essentially of, or yet further consisting of a crystalline DMXAA sodium salt in form of an anhydrate or a solvate; and

one or more further pharmaceutically active compounds selected from taxanes, platins, cyclophosphamide, vinca alkaloids, antimetabolites, topoisomerase II inhibitors, anthracyclines, tumour necrosis factor (TNF) stimulating compounds, immunomodulatory compounds, non steroidal anti-inflammatory drugs (NSAIDs), EGFR signalling pathway inhibitors and VEGF binders.

Alternatively, there is provided a pharmaceutical formulation comprising, or alternatively consisting essentially of, or yet further consisting of a crystalline DMXAA sodium salt in the form of an anhydrate or a solvate with a physiologically acceptable solvent and one or more further pharmaceutically active compounds selected from the list in (b) above.

Embodiments of the above-defined kit that may be mentioned include those in which formulation (a) above (i.e. that containing DMXAA) is a formulation that is adapted for intravenous injection (e.g. an aqueous solution). Alternatively, and in a separate embodiment, formulation (a) may contain the Crystalline DMXAA sodium salt in solid form.

In a particular embodiment of the invention, the one or mere separate formulations mentioned at (b) above may be a formulation containing paclitaxel, and, separately, a formulation containing carboplatin. Each of these formulations may in particular embodiments, be a formulation adapted for intravenous injection.

In another particular embodiment, the one or more separate formulations is a formulation (e.g. a formulation adapted for intravenous Injection) containing docetaxel.

A particular embodiment of a crystalline form of DMXAA sodium, salt according to the invention is a hydrate containing about 20-22% water and exhibiting a characteristic X-ray powder diffraction pattern with characteristic peaks expressed in d-values (Å):

102 (s), 9.3 (m), 3.54 (vs), and 3.19 (vs),
hereinafter designated as form B.

In certain embodiments, form B exhibits a characteristic X-ray powder diffraction pattern with characteristic peaks expressed in d-values (Å):

11.1 (w), 10.2 (s), 9.3 (m), 7.0 (m), 6.5 (m), 5.57 (m), 3.62 (s), 3.54 (vs), 3.38 (m), and 3.19 (vs).

In more particular embodiments, form B exhibits a characteristic X-ray powder diffraction pattern with characteristic peaks expressed in d-values (Å):

11.1 (w), 10.2 (s), 9.3 (m), 7.0 (m), 6.5 (m), 557 (m), 5.41 (w), 5.21 (m), 5.04 (w), 4.67 (w), 4.53 (m), 4.29 (w), 4.25 (w), 4.12 (w), 4.05 (vw), 3.75 (m), 3.69 (w), 3.62 (s), 3.54 (vs), 3.38 (m), 3.24 (m), 3.19 (vs), anti 3.16 (m).

Here and in the following the abbreviations in brackets mean: (vvs)=very very strong intensity; (vs)=very strong intensity; (s)=strong intensity; (m)=medium intensity; (w)=weak intensity and (vw)=very weak intensity.

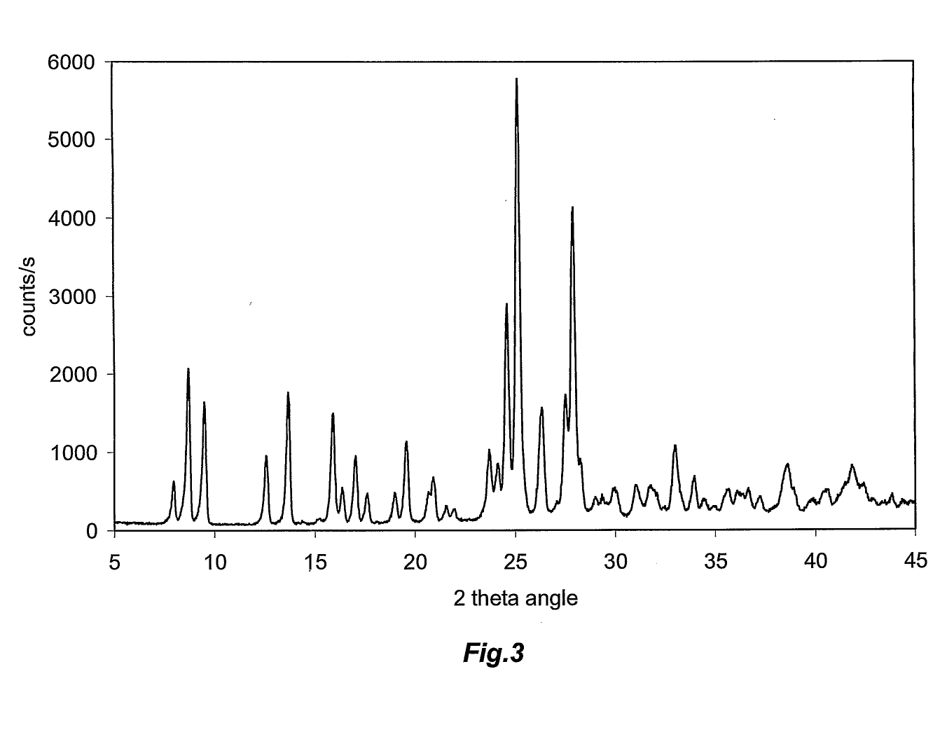

In yet further embodiments, form B exhibits a characteristic X-ray powder diffraction pattern essentially as exhibited in FIG. 3 when powder X-ray diffraction is carried out using Cu K_{α} radiation.

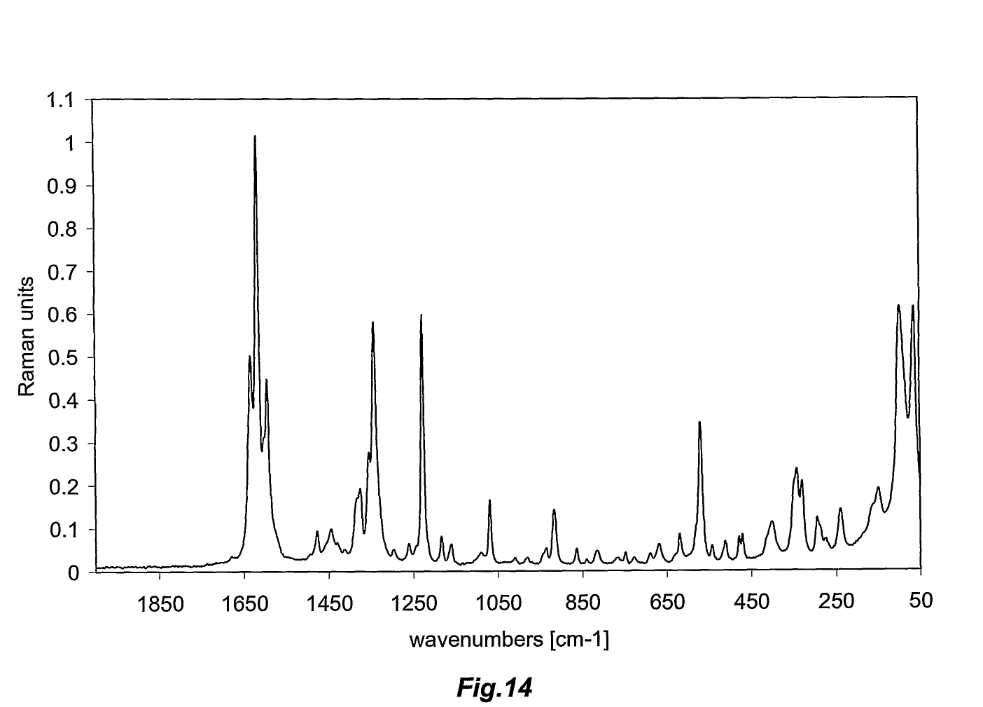

In yet further embodiments, form B exhibits a characteristic Raman spectrum essentially as exhibited in FIG. 14, with the most prominent peaks at 16.33, 1617, 1594; 1375, 1342, 1228, 1069, 570, 98, and 64 cm⁻¹.

The aqueous solubility of form B is at least 300 mg/mL at 22° C. at pH 8.3.

DMXAA sodium salt form B is prepared by a process comprising stirring a suspension of an amorphous form or any crystalline form of DMXAA sodium salt in an organic solvent containing water as a co-solvent in a ratio that results in A water activity of about 0.6 to 0.95, such as from 0.7 to 0.9, filtering off the obtained crystalline form and drying it under moderate conditions, such as under a relative humidity of about 75%.

Surprisingly, it has been found that form B exhibits physical stability under a high relative humidity. A high relative humidity is here defined as a relative humidity of about 70 to 90%. Tests carried out at 40° C. have shown that form B is chemically and physically very stable under such conditions. In particular, the water content can be well controlled and remains constant, over time. Therefore, form a can be produced in a well controlled manner from water solvent mixtures with a water activity of about 0.7 to 0.9.

When kept in a tight container, form B shows chemical/physical stability (no change observed after 36 months storage at ambient temperature in a tight PE packaging).

Another particular embodiment of a crystalline form of DMXAA sodium salt according to the invention is a hydrate containing about 15-20% water and, exhibiting a characteristic X-ray powder diffraction pattern with characteristic peaks expressed in d-values (Å):

10.2 (vs), 8.7 (s), 5.60 (s), and 3.67 (vs)
hereinafter designated as form C.

In certain embodiments, form C exhibits a characteristic X-ray powder diffraction pattern with characteristic peaks expressed in d-values (Å):

11.2 (M), 10.2 (vs), 9.0 (w), 8.7 (s), 5.60 (s), 3.67 (vs), 3.54 (m), 3.49 (vs), 3.40 (s), 3.32 (s), and 3.26 (vs).

In particular embodiments, form C exhibits a characteristic X-ray powder diffraction pattern with characteristic peaks expressed in d-values (Å):

11.2 (m), 102 (vs), 90 (w), 8.7 (s), 6.9 (w), 67 (s), 6.5 (w), 6.4 (vw), 5460 (s), 5.52 (m), 5.16 (m), 4.97 (m), 4.44 (m), 4.36 (w), 4.25 (w), 4.07 (w), 3.67 (vs), 3.54 (m), 3.49 (vs), 3.40 (s), 3.32 (s), 3.26 (vs), 3.20 (m), 3.09 (m), 3.03 (m), 2.80 (m), and 2.63 (m).

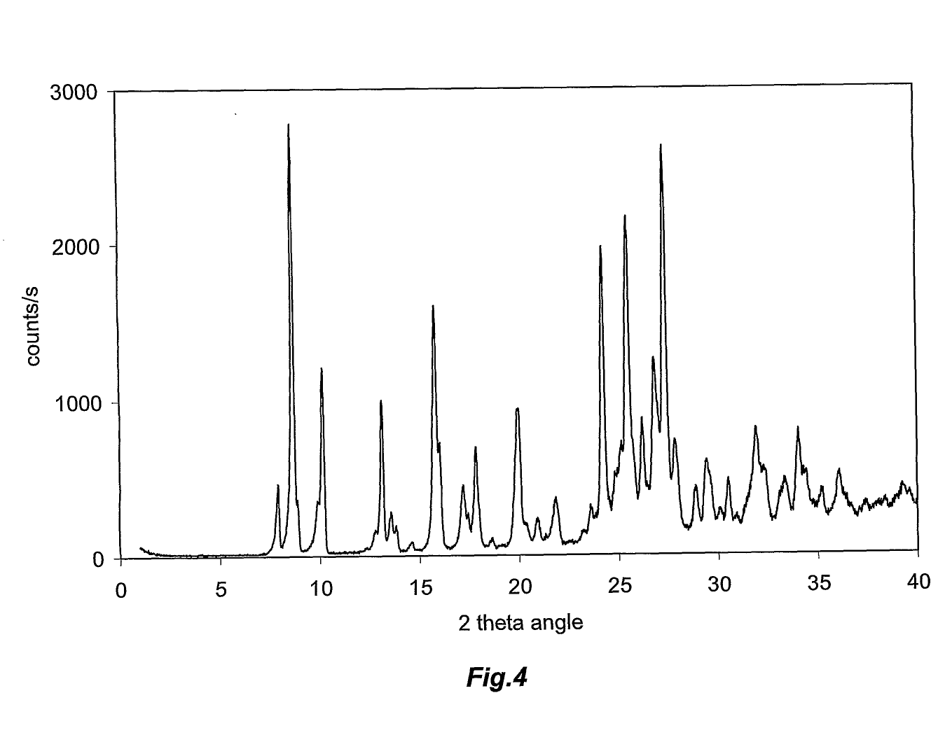

In yet further embodiments, form C exhibits a characteristic X-ray powder diffraction pattern essentially as exhibited in FIG. 4 when powder X-ray diffraction is carried out using Cu K_{L} radiation.

In yet further embodiments, form C exhibits a characteristic Raman spectrum essentially as exhibited in FIG. 15, with the most prominent peaks at 1613, 1586, 1343, 1228, 1066, 340, and 91 cm⁻¹.

The aqueous solubility of form C is at least 300 mg/mL at 22° C. at pH 8.3.

DMXAA sodium salt form C is prepared by a process comprising stirring a suspension of an amorphous form or any crystalline form of DMXAA sodium salt in an organic solvent (e.g. a solvent selected from the group comprising acetone, ethyl acetate, tetrahydrofuran and binary or ternary mixtures thereof) containing water as a co-solvent in a ratio that results in a water activity of about 0.2 to 0.7, such as from 0.4 to 0.6, filtering off the obtained crystalline form and drying it under moderate conditions, such as under a relative humidity of about 50%.

Surprisingly, it has been found that form C exhibits a physical stability under an intermediate relative humidity. An intermediate relative humidity is here defined as a relative humidity of about 20 to 60%. In particular, the water content can be well controlled and remains constant over time when DMXAA sodium salt is exposed to such humidity conditions. Therefore, form C can be produced in a well controlled manner from water solvent mixtures with a water activity of about 0.2 to 0.6.

When kept in a tight container, form C shows chemical/physical stability (no change observed after 36 months storage at ambient temperature in a tight PE packaging).

It should be noted that forms B and C might be regarded as isomorphic hydrates both exhibiting essentially the same crystal lattice with small changes of crystal parameters.

Another embodiment of a crystalline form of DMXAA sodium salt according to the invention is a hydrate containing about 23-30% water and exhibiting a characteristic X-ray powder diffraction pattern with characteristic peaks expressed in d-values (Å):

12.6 (s), 11.7 (vs), 8.1 (m), 6.3 (m), 5.94 (m), 5.64 (m), and 3.57 (s)
hereinafter designated as form D.

In certain embodiments, form D exhibits a characteristic X-ray powder diffraction pattern with characteristic peaks expressed in d-values (Å):

12.6 (s), 11.7 (vs), 9.2 (vw), 8.1 (m), 6.3 (m), 5.94 (m), 5.64 (m), 5.07 (w), 4.65 (w), 3.91 (w), 3.87 (w), 3.82 (w), 3.72 (w), 3.57 (s), 3.24 (m), 3.19 (m), 3.11 (s), 3.05 (w), and 2.79 (m).

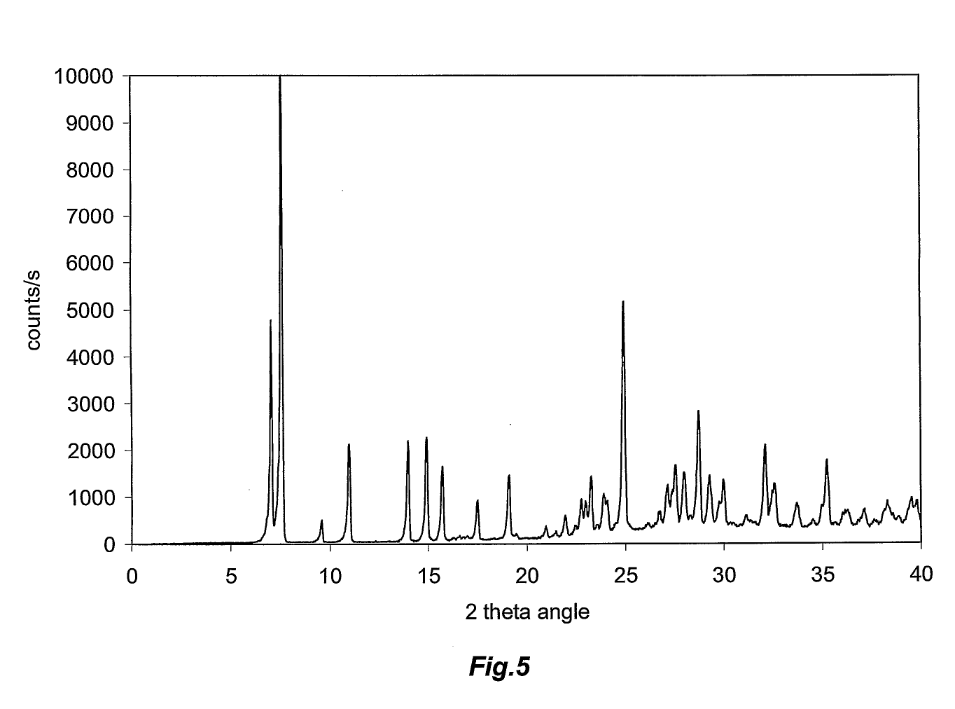

In particular embodiments, form D exhibits at a wavelength of 1.54060 Å a characteristic X-ray powder diffraction pattern essentially as exhibited in FIG. 5.

DMXAA sodium salt form D can be prepared by suspending a mixture of DMXAA sodium salt forms B and C in a TRIS/acetate buffer and recovering the solid by filtration.

Another embodiment of a crystalline form, of DMXAA sodium salt according to the invention is a hydrate containing about 7-9% water and exhibiting a characteristic X-ray powder diffraction pattern with characteristic peaks expressed in d-values (Å):

16.2 (vs), 13.2 (s), 11.1 (s), 9.6 (vs), 8.1 (s), 6.5 (vs), and), 5.43 (s), hereinafter designated as form H.

In certain embodiments, form H exhibits a characteristic X-ray, powder diffraction pattern with characteristic peaks expressed in d-values (Å):

16.2 (vs), 14.5 (w), 13.2 (s), 11.1 (s), 9.6 (vs), 8.1 (s), 74 (w), 6.6 (s), 6.5 (vs), 6.1 (w), 5.83 (m), 5.43 (s), 4.81 (m), 4.41 (m), 4.29 (w), 3.63 (m), 3.58 (m), 3.45 (m), 3.31 (m), 3.03 (m), 2.77 (w), and 2.59 (w).

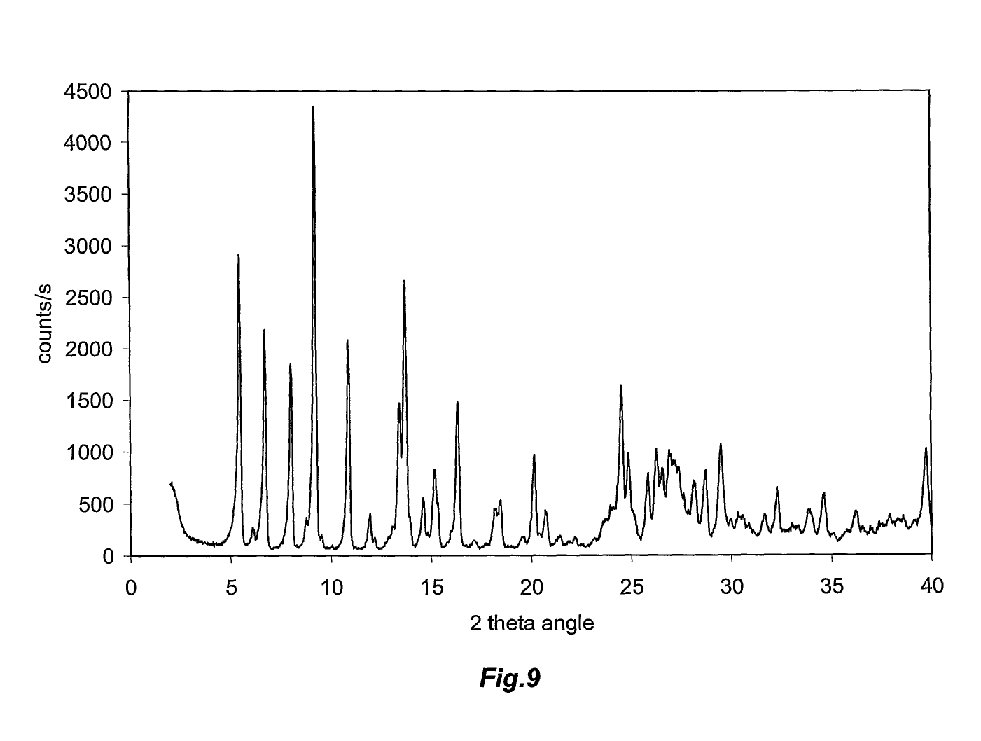

In particular embodiments, form H exhibits a characteristic X-ray powder diffraction pattern essentially as exhibited in FIG. 9 when powder X-ray diffraction is carried out using Cu Kₐ radiation.

DMXAA sodium salt form H can be prepared by suspending DMXAA sodium salt form C in ethanol and recovering the solid by filtration, or suspending DMXAA in acetone, adding a sodium hydroxide solution, stirring and recovering the solid by filtration.

Another embodiment of a crystalline form of DMXAA sodium salt according to the invention is a hydrate containing about 7-9% water and exhibiting a characteristic X-ray powder diffraction pattern with characteristic peeks expressed in d-values (Å):

14.6 (vs), 9.7 (vs), 7.3 (s), 5.88 (O), 3.64 (s), 3.59 (s), 3.26 (s), and 3.22 (s),
hereinafter designated as form I.

In certain embodiments, form I exhibits a characteristic X-ray powder diffraction pattern with characteristic peaks expressed in d-values (Å):

14.6 (vs), 11.1 (m), 9.7 (vs), 8.9 (w), 7.3 (a), 6.9 (m), 6.5 (w), 6.2 (m), 6.1 (w), 5.83 (m), 5.80 (m), 5.63 (w), 5.16 (m), 5.02 (w), 4.31 (m), 3.72 (m), 3.64 (s), 3.59 (s), 3154 (m), 346 (m), 3.41 (m), 3.26 (s), 3.22 (s), 3.02 (m), and 2.91 (m).

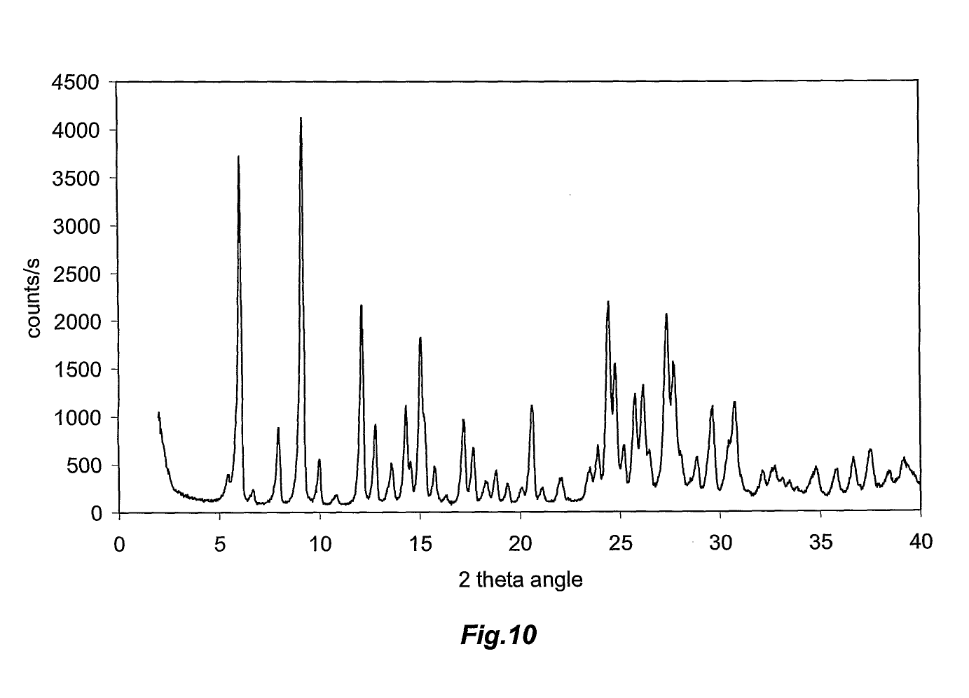

In particular embodiments, form I a characteristic X-ray powder diffraction pattern essentially as exhibited in FIG. 10 when powder X-ray diffraction is carried out using Cu K_{α} radiation, DMXAA sodium salt form I can be prepared by suspending DMXAA sodium salt form B in ethanol and recovering the solid by filtration.

Another particular embodiment of a crystalline form of DMXAA sodium salt according to the invention is an anhydrate exhibiting a characteristic X-ray powder diffraction pattern with characteristic peaks expressed in d-values (Å):

9.7 (m), 90 (m), and 3.48 (vs)
hereinafter designated as form F.

In certain embodiments, form F exhibits a characteristic X-ray powder diffraction pattern with characteristic peaks expressed in d-values (Å):

11.7 (m), 9.7 (m), 9.0 (m), 5.56 (m), 3.93 (m), and 0.48 (vs).

In particular embodiments, form F exhibits a characteristic X-ray powder diffraction pattern with characteristic peaks expressed in d-values (A):

11.7 (m), 9.7 (m), 9.0 (m), 8.5 (vw), 7.1 (w), 68 (w), 6.5 (vw), 6.0 (vw), 5.86 (w), 5.56 (m), 5.37 (vw), 5.07 (m), 4.76 (w), 4.45 (w), 3.93 (m), 3.72 (w), 3.56 (m), 3.48 (vs), 3.20 (w), 2.95 (m), and 2.91 (m).

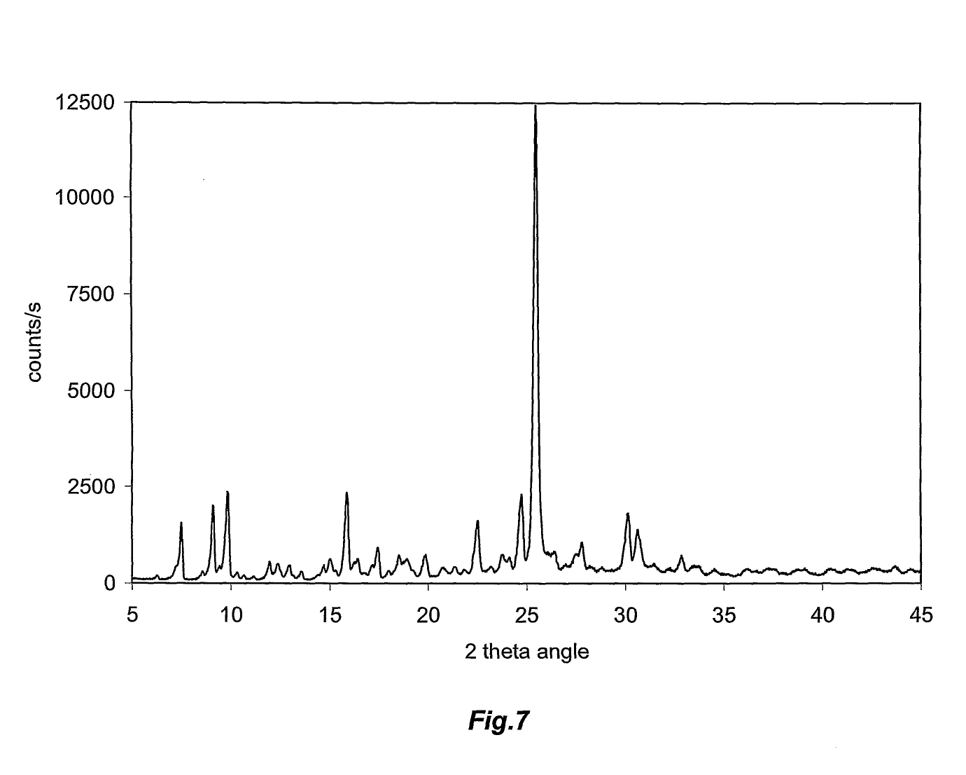

In yet further embodiments, form F exhibits at wavelength of 1.54060 Å a characteristic X-ray powder diffraction pattern essentially as exhibited in FIG. 7.

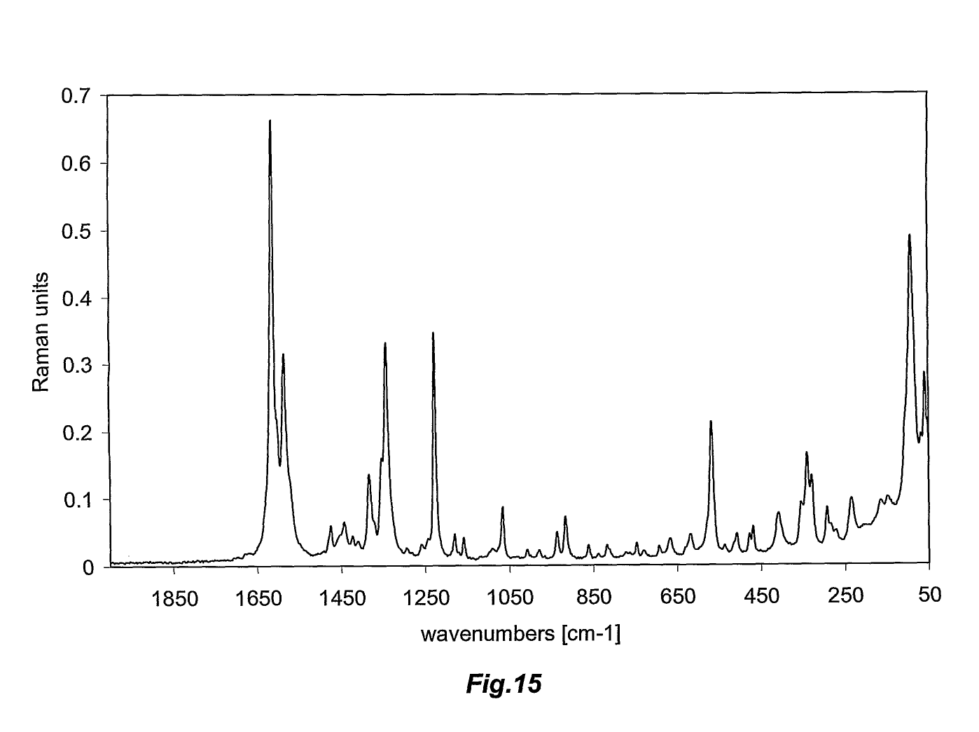

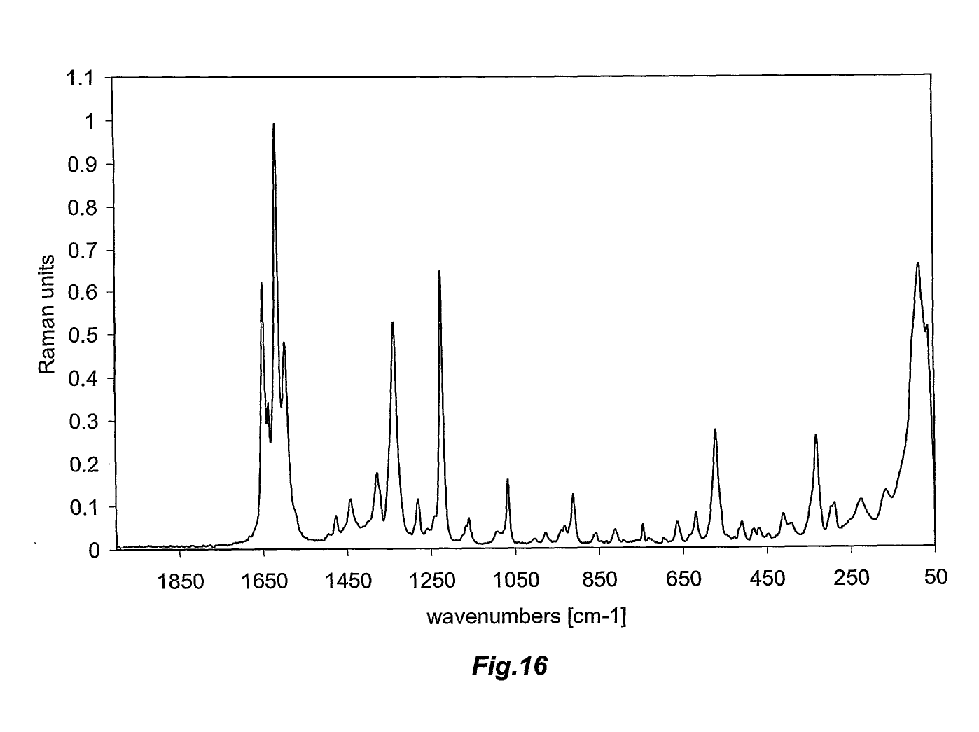

In yet further embodiments, form F exhibits a characteristic Raman spectrum essentially as exhibited in FIG. 16, with the most prominent peaks at 1650, 1817, 1598, 1339, 1226, 1068, 572, 322, and 85 cm⁻¹.

The aqueous solubility of form F is at least 300 mg/mL at 22° C. at pH 8.3.

Form F is the stable anhydrate known of DMXAA sodium salt. Phase, equilibration experiments show in particular that Form F is more stable than hereafter described form E or form L.

Form F is hygroscopic under standard laboratory conditions and when exposed to a relative humidity over 70% or a water activity over 0.70 is prone to transform into form B or form C.

When kept in a tight container, form F shows chemical/physical stability (no change observed after 36 months storage at ambient temperature in a tight PE packaging).

DMXAA sodium salt form F can be prepared by a process comprising suspending an amorphous or any crystalline form of DMXAA sodium salt in an essentially water-free organic solvent (e.g. a solvent selected from the group comprising 2-butanone and isopropanol) and stirring, obtained suspension until formation of form F is complete, filtering and drying in dry air at ambient temperature.

Another embodiment of a crystalline form of DMXAA sodium salt according to the invention is an anhydrate exhibiting a characteristic X-ray powder diffraction pattern with characteristic peaks expressed in d-values (Å):

17.4 (vs), 14.1 (m), 8.9 (vs), 8.7 (vs), and 5.76 (s)
hereinafter designated as form E.

In certain embodiments, form E exhibits a characteristic X-ray powder diffraction pattern with characteristic, peaks expressed in d-values (Å):

17.4 (vs), 14.1 (m), 11.5 (m), 10.4 (m), 10.1 (s), 8.9 (vs), 8.7 (vs), 6.5 (s), 6.3 (w), 5.76 (s), and 3.49 (m, broad).

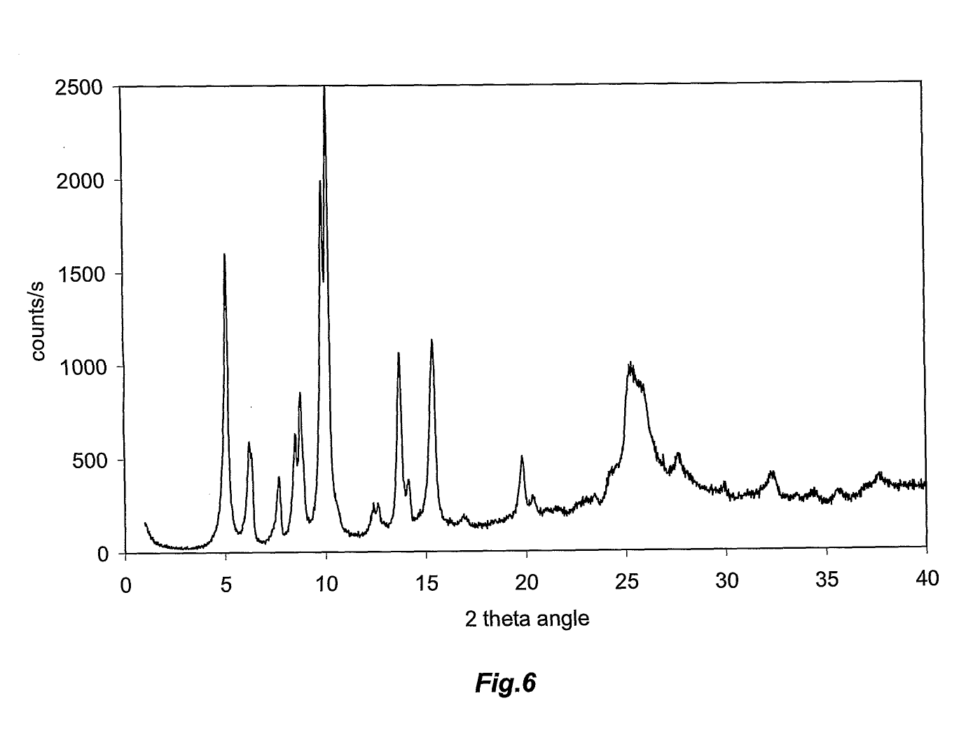

In particular embodiments, form E exhibits a characteristic X-ray powder diffraction pattern essentially as exhibited in FIG. 6 when powder X-ray diffraction is carried out using Cu K_{α} radiation.

DMXAA sodium salt faun E can be prepared by storing a mixture of DMXAA sodium salt forms B and C for a sufficient period of time in a dry atmosphere.

Another embodiment of a crystalline form of DMXAA sodium salt according to the invention is an anhydrate exhibiting a characteristic X-ray powder diffraction pattern with characteristic peaks expressed in d-values (Å):

7.8 (vs), 6.8 (s), 5.76 (s), 3.94 (s), 3.78 (w), and 3.60 (vs),
hereinafter designated as form L.

In certain embodiments, form L exhibits a characteristic X-ray powder diffraction pattern with characteristic peaks expressed in d-values (Å):

13.6 (w), 11.5 (m), 9.3 (m), 8.7 (w), 7.8 (vs), 7.3 (m), 6.8 (s), 6.0 (m), 5.87 (w), 5.76 (s), 4.58 (m), 4.45 (m); 4.40 (m), 4.24 (m), 4.20 (m), 3.94 (s), 3.84 (w), 378 (w), 3.60 (vs), 3.38 (vs), 3.00 (m), and 2.89 (m).

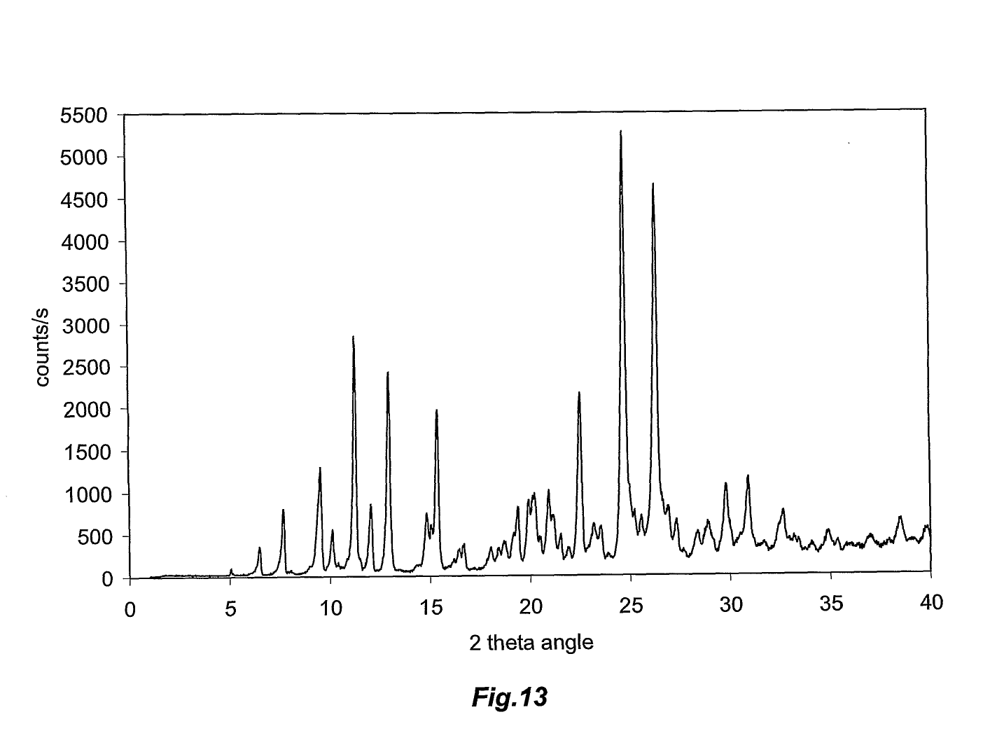

In particular embodiments, form L exhibits a characteristic X-ray powder diffraction pattern essentially as exhibited in FIG. 13 when powder X-ray diffraction is carried out using Cu K_{α} radiation.

DMXAA sodium salt form L can be prepared by suspending DMXAA sodium salt form K in tetrahydrofuran, filtering offend drying the obtained solid under a flow of dry nitrogen.

Another embodiment of a crystalline form of DMXAA sodium salt according to the invention is an ethanol solvate containing about 21-22% ethanol and exhibiting a characteristic X-ray powder diffraction pattern with characteristic peaks expressed in d-values (Å):

12.3 (vs), 10.4 (s), 6.8 (m), 6.1 (m), and 3.42 (m),
hereinafter designated as form K.

In certain embodiments, form K exhibits a characteristic X-ray powder diffraction pattern with characteristic peeks expressed in d-values (Å):

14.7 (vw), 12.3 (vs), 104 (s), 6.8 (m), 6.1 (m), 5.46 (w), 5.34 (m), 5.16 (w), 5.08 (m), 4.72 (w), 4.64 (w), 4.29 (w), 3.98 (w), 3.80 (w), 366 (w), 3.57 (m), 3.48 (w), 3.42 (m), 3.27 (m), and 2.94 (m).

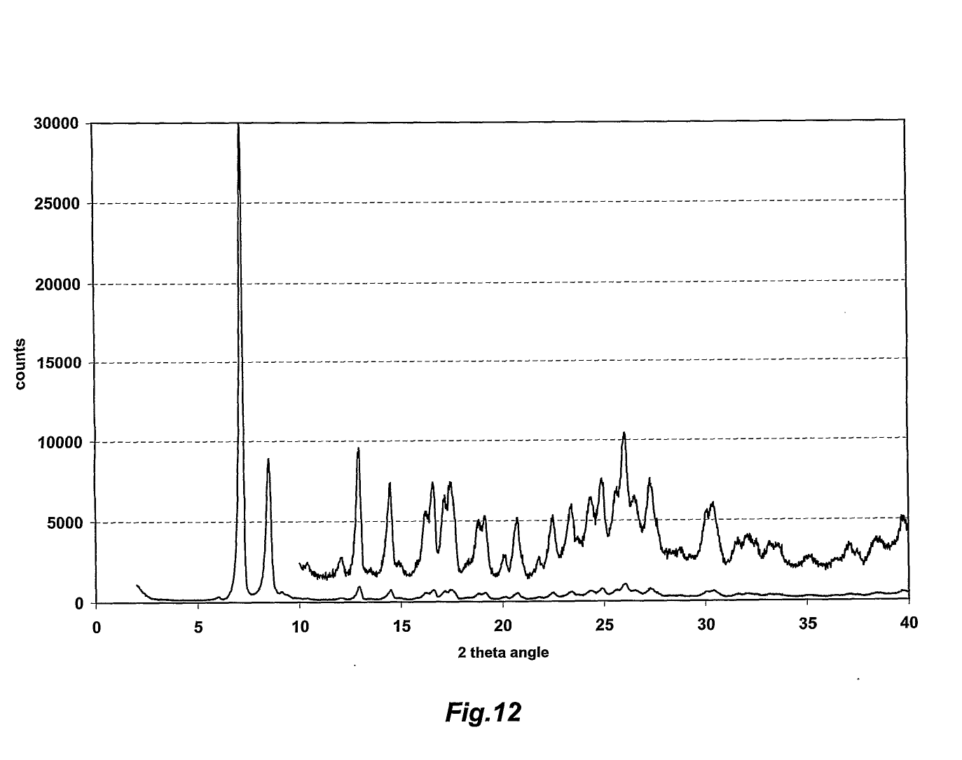

In particular embodiments, form K exhibits a characteristic X-ray powder diffraction pattern essentially as exhibited in FIG. 12 when powder X-ray diffraction is carried out using Cu K_{α} radiation.

DMXAA sodium salt form K can be prepared by suspending an amorphous form of DMXAA sodium salt in ethanol, filtering off and drying the obtained solid under a dry nitrogen flow.

Another embodiment of a crystalline form of DMXAA sodium salt according to the invention is a mixed hydrate-isopropanol solvate containing about 10-11% water and about 10-11% isopropanol and exhibiting a characteristic X-ray powder diffraction pattern with characteristic peaks expressed in d-values (Å):

17.0 (s), 12.0 (s), 8.5 (s) and 5.65 (s),
hereinafter designated as form G.

In certain embodiments, form G exhibits a characteristic X-ray powder diffraction pattern with characteristic peaks expressed in d-values (Å):

17.0 (s), 12.0 (s), 10.7 (m), 8.6 (s), 7.6 (m), 6.0° (m), 5.82 (m), 5.65 (s), 4.70 (m), 4.45 (m), 4.37 (m), 3.32 (vs), and 3.21 (s).

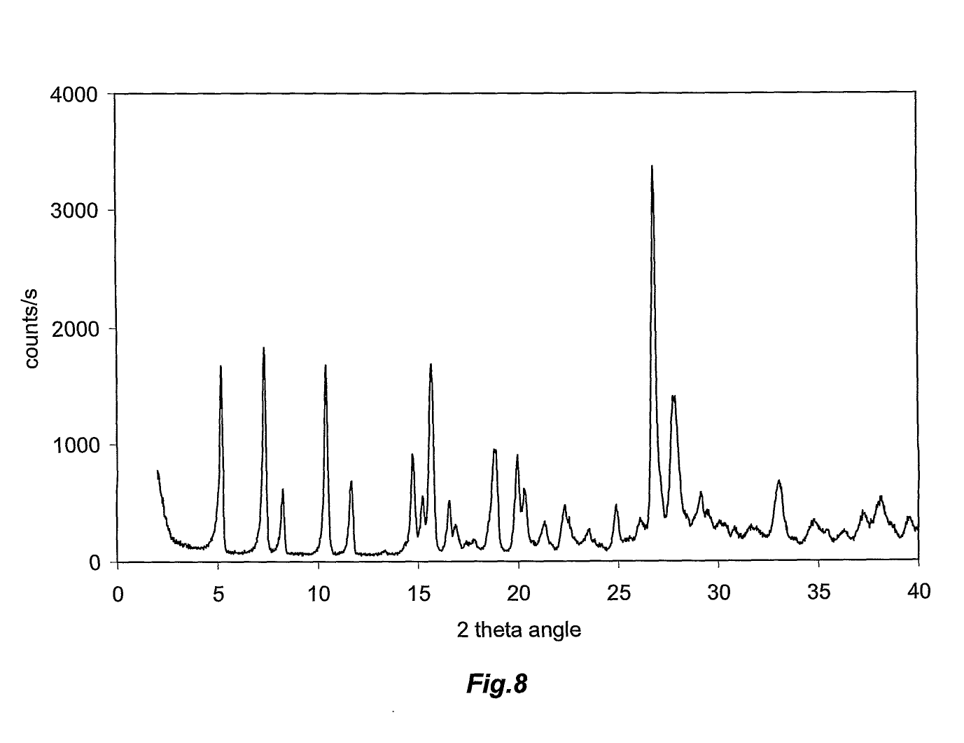

In particular embodiments, form G exhibits a characteristic X-ray powder diffraction pattern essentially as exhibited in FIG. 8 when powder X-ray diffraction is carried out using Cu K_{α} radiation.

DMXAA sodium salt form G can be Obtained by dissolving an amorphous form of DMXAA sodium salt in water, adding isopropanol, Stirring, filtering Off and drying the obtained solid in dry air.

Each of the different crystalline forms of DMXAA Sodium salt described herein can be used in medical therapy, notably in the treatment of cancer, as an active substance in a solid pharmaceutical composition, or as an easy to handle process ingredient for preparing a liquid pharmaceutical composition containing dissolved DMXAA sodium salt as active substance.

In some embodiments, the forms suitable for the pharmaceutical use disclosed herein, are crystalline forms B and C of DMXAA sodium salt hydrate, which are the stable hydrates and are interconvertible into one another, depending on the water activity, and crystalline form F of DMXAA sodium salt anhydrate which is the stable anhydrate. Each of those forms shows chemical/physical stability (no change observed after 36 months storage at ambient temperature in a tight PE packaging) and water solubility (at least 100 mg/mL at 22° C. at pH 8.3).

As well as being relevant to the pharmaceutical use described herein, crystalline forms D, H and I of DMXAA sodium salt hydrate and crystalline form K of DMXAA sodium salt ethanol solvate can be used as intermediates for preparing hydrate forms B and C.

Similarly, crystalline forms E and L of DMXAA sodium salt anhydrate and crystalline form K of DMXAA sodium salt ethanol solvate can be used as intermediates for preparing anhydrate form F.

### Pharmaceutical Compositions

The crystalline DMXAA sodium salt of the present invention may be administered alone or may be administered as a pharmaceutical composition or formulation—e.g. when the components are in admixture with a suitable pharmaceutical excipient, diluent or carrier selected with regard to the intended route of administration. The pharmaceutical compositions will typically comprise any one or more of a pharmaceutically acceptable diluent, carrier, or excipient. Acceptable carriers or diluents for therapeutic use are well known in the pharmaceutical art, and are described, for example, in Remington's Pharmaceutical Sciences, Mack Publishing Co. (A. R. Gennaro edit 1985). The pharmaceutical compositions may comprise as—or in addition to—the carrier, excipient or diluent any suitable binder(s), lubricant(s), suspending agent(s), coating agent(s), solubilising agent(s), buffers, flavouring agents, surface active agents, thickeners, preservatives (including anti-oxidants) and the like, and substances included for the purpose of rendering the formulation isotonic with the blood of the intended recipient.

Examples of suitable carriers include, but are not limited to, lactose, starch, glucose, methyl cellulose, magnesium stearate, mannitol, sorbitol and the like. Examples of suitable diluents include, but are not limited to, ethanol, glycerol and water.

Examples of suitable binders include, but are not limited to, starch, gelatin, natural sugars such as glucose, anhydrous, lactose, free-flow lactose, beta-lactose, corn sweeteners, natural and synthetic gums, such as acacia, tragacanth or sodium alginate, carboxymethyl cellulose and polyethylene glycol.

Examples of suitable lubricants include, but are not limited to, sodium oleate, sodium stearate, magnesium stearate, sodium benzoate, sodium acetate, sodium chloride and the like.

Preservatives, stabilizers, dyes and even flavouring agents may be provided in the pharmaceutical composition. Examples of preservatives include, but are not limited to, sodium benzoate, sorbic acid and esters of p-hydroxybenzoic acid. Antioxidant and suspending agents may be also used.

### Pharmaceutical Compositions for Oral Administration

if the pharmaceutical composition is a tablet or a pill, then it may contain excipients such as, but not limited to, microcrystalline cellulose, lactose, sodium citrate, calcium carbonate, dibasic calcium phosphate and glycine, disintegrants such as starch (preferably corn, potato or tapioca starch), sodium starch glycollate, croscarmellose sodium and certain complex silicates, and granulation binders such as polyvinylpyrrolidone, hydroxypropylmethylcellulose (HPMC), hydroxypropylcellulose (HPC), sucrose, gelatin and acacia. Additionally, lubricating agents such as, but not limited to, magnesium stearate, stearic acid, glyceryl behenate and talc may be included.

Solid compositions of a similar type may also be employed as fillers in gelatin capsules. Excipients that may be mentioned in this regard include, but are not limited to, lactose, starch, cellulose, milk sugar or high molecular weight polyethylene glycols. For aqueous suspensions and/or elixirs, the compound may be combined with various sweetening or flavouring agents, colouring matter or dyes, with emulsifying and/or suspending agents and with diluents such as, but not limited to, water, ethanol, propylene glycol and glycerin, and combinations thereof.

### Formulations for Intravenous Injection

A pharmaceutical formulation for intravenous injection containing as active substance DMXAA sodium salt may be prepared by dissolving a suitable amount of crystalline DMXAA sodium salt in an aqueous solvent. If necessary, pH adjustment may be used to control the solubility of DMXAA in the aqueous solvent.

For example a pharmaceutical formulation for intravenous injection containing as active substance 200 mg/mL DMXAA sodium salt is prepared by dissolving a suitable amount of crystalline DMXAA sodium salt in an aqueous solvent having a physiologically acceptable pH, e.g. a 0.01M Ins buffer solution, and adjusting the pH to 7.8-8.6 by addition of hydrochloric acid.

If desired, the formulation for intravenous injection may be diluted (e.g. with water or 5% solution of glucose in water) prior to injection.

The following examples illustrate the invention without limiting its scope.

### EXAMPLES

In the examples and elsewhere, abbreviations have the following meanings:

| | |
|---|---|
| a_{w} | water activity |
| Å | angstrom |
| cm | centimeter |
| DMSO | dimethylsulfoxide |
| DSC | Differential Scanning Calorimetry |
| DVS | Dynamic Vapour Sorption |
| Kg | kilogram |
| μm | micrometer |
| μl | microliter |
| mg | milligram |
| m | meter |
| M | molar |
| mL | milliliter |
| NaHCO₃ | sodium bicarbonate |
| nm | nanometer |
| ppm | parts per million |
| PXRD | Powder X-ray Diffraction |
| r.h. | relative humidity |
| TG-FTIR | Thermogravimetry coupled with FT |
| | IR spectroscopy |
| THF | tetrahydrofuran |
| v/v | volume by volume |

### EXPERIMENTAL

Powder X-ray Diffraction (PXRD): PXRD is performed on a Philips 1710 powder X-ray diffractometer using CuK_{α} radiation (both K_{α1} and K_{α2} with a ratio of 2:1). Powder X-ray diffraction measurements were carried out in reflectance mode. d-spacings are calculated from the 2θ values using the wavelength of 1.54060 Å. Generally, 2θ values are within an error of ±0.1 to ±0.2°. The experimental error on the d-spacing values is therefore dependent on the peak location.

Raman spectroscopy: FT-Raman spectra are recorded on a Bruker RFS 100 FT-Raman system with a near infrared Nd:YAG laser operating at 1064 nm and a liquid nitrogen-cooled germanium detector. For each sample, 64 scans with a resolution of 2 cm⁻¹ are accumulated. Generally, 100 mW laser power is used.

DSC: Differential scanning calorimetry was carried out with a Perkin Elmer DSC7 (closed gold sample pan or gold-plated steel sample pan, heating rate 10 K/min or 20 K/min).

TG-FTIR: Thermogravimetric measurements were carried out with a Netzsch Thermo-Microbalance TG 209 coupled to a Broker FTIR Spectrometer Vector 22 (sample pans with a pinhole, N₂ atmosphere, heating rate 10 K/min).

DVS: Dynamic vapour sorption measurements were carried out with an instrument of Surface Measurement Systems (http:www.smsuk.co.uk). The weight change of the investigated sample is monitored while the relative humidity is changed over a range from 0% r.h. to 95% r.h. at a change rate of 5% per hour.

Water activity (a_{w}): The water activity is a thermodynamic parameter which is related to the chemical potential of Water in a reacting system. In the gas phase, i.e., in air, the water activity essentially corresponds to the relative humidity in % divided by 100. For instance a relative humidity of 75% corresponds to a water activity of 0.75 it is well known that the water activity in a mixture of an organic solvent with water depends upon the water concentration and the miscibility of the solvent with water, generally in a non-linear way. Such water activities are known from chemical and physical reference data tables. For instance in D. R. Lide, CRC handbook of thermophysical and thermochemical data 1994.

### A) Preparation of DMXAA and a Crystalline Form of DMXAA Sodium Salt Methanol Solvate

### Reference Example

Purified DMXAA was prepared according to the method described by G. W. Rewcastle, J. Med. Chem. 1991, 34, 217-222, including the following steps:

(a) condensation of 2,3-dimethylaniline with hydroxylamine and chloral hydrate to give 2,3-dimethyl-α-isonitrosoacetanilide,
(b) sulphuric acid-catalysed ring closure to give 6,7-dimethylisatin,
(c) oxidative ring opening using hydrogen peroxide in potassium hydroxide solution to give 3,4-dimethylanthranilic acid,
(d) treatment of the latter acid with sulfuric acid, sodium nitrate and potassium iodide to give 3,4-dimethyl-2-iodobenzoic acid,
(e) stirring the latter acid with the disodium salt of 2-hydroxyphenylacetic acid, tris[2-(2-methoxyethoxy)ethyl]amine and copper monochloride in DMSO,
(f) evaporation, and treatment with an acetic acid solution to, give 2-[(2-carboxymethyl)phenoxy]-3,4-dimethylbenzoic acid, and
(g) treatment of the latter acid with concentrated sulfuric acid, washing with water and crystallisation in methanol to give purified DMXAA.

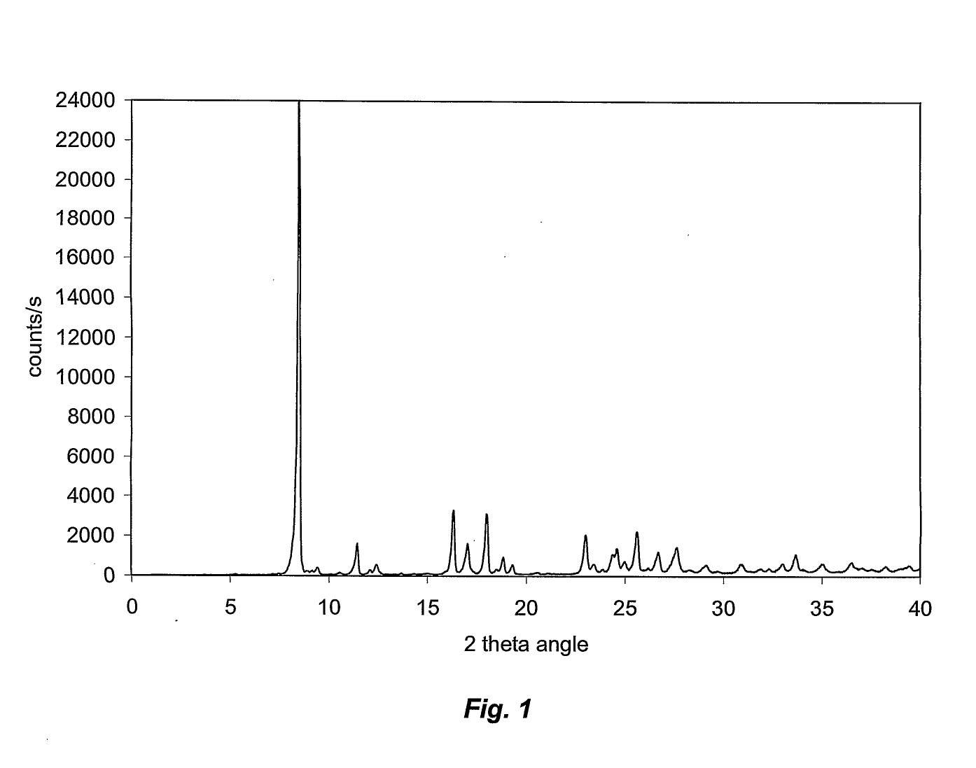

10 g of purified, DMXAA were dissolved in water containing one equivalent of NaHCO₃. The obtained solution was filtered through a P4 glass filter and thereafter obtained filtrate was evaporated to dryness using a rotary evaporator. The residue in the glass flask was dissolved with 100 mL of hot methanol and DMXAA sodium salt was recrystallised from the hot solution by addition of 100 mL of ethyl acetate and cooling to 10° C. The obtained crystalline product was isolated by filtration and drying in under nitrogen at room temperature. Powder X-ray diffraction of this sample showed that a crystalline form was obtained which exhibits a powder pattern as shown in FIG. 1. This form is designated as form M (or crystalline form M). Investigation of form M by TG-FTIR shows that it contains about 17% of methanol.

### B) Preparation of Mesomorphic Form A of DMXAA Sodium Salt

### Example A1

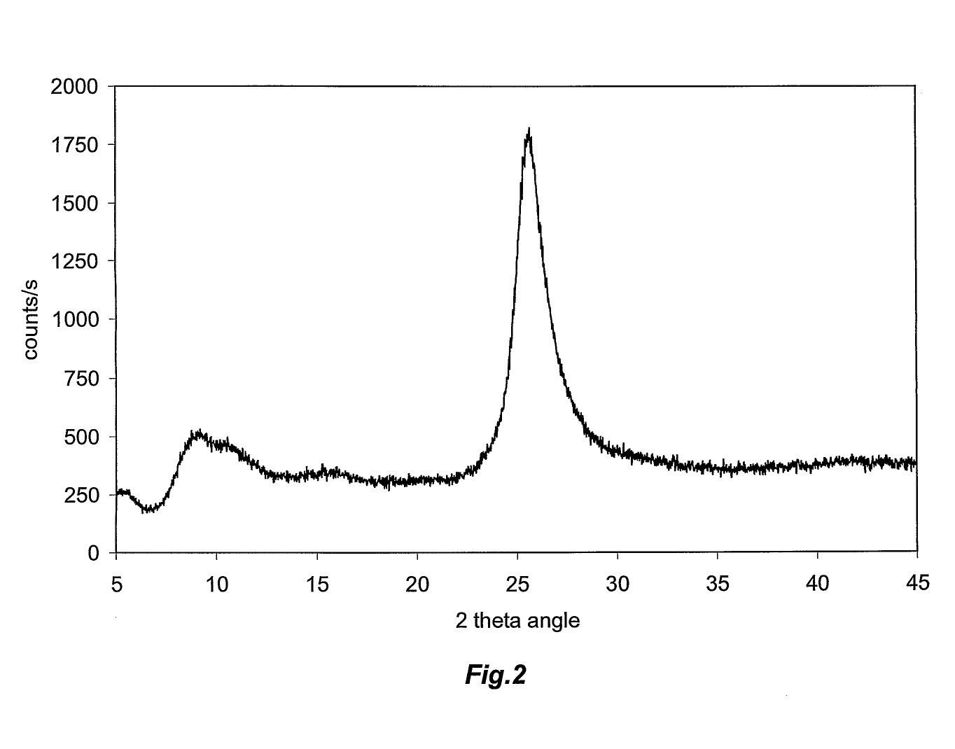

897 mg of DMXAA sodium salt methanol solvate crystalline form M, according to the Reference Example, are dissolved in 4.0 mL of water at room temperature. This solution is filtered through a 0.22 μm Millipore filtration unit into a 250 mL round flask, wherein the solution is frozen at −78° C. with solid CO₂ (dry ice). Thereafter, freeze drying was performed with a lyophilizer model Christ Beta 2-8, L9-2 using a cooler temperature of −89° C. with a resulting pressure of 0.090 mbar. After about 18 hours of freeze drying, a dry powder was obtained which was investigated by powder X-ray diffraction and Raman spectroscopy. The powder X-ray diffraction pattern shows, one single distinct peak at 2θ=25.7° which is typical for a mesomorphic or amorphous form, here-designated as form A (or mesomorphic form A). A powder X-ray diffraction pattern of form A is shown in FIG. 2.

Stability: When stored under nitrogen in a tightly sealed container, mesomorphic form A remains stable for at least two months at ambient temperature.

### C) Preparation and Characterization of Crystalline Forms of DMXAA Sodium Salt According to the Invention

### 1) Preparation and Characterization of Form B

### Example B1

### Preparation of Form B from Mesomorphic Form A

About 50 mg of DMXAA sodium salt mesomorphic form A, according to example A1, are stored in a humidity chamber with 75% relative humidity (corresponding to a water activity of 0.75) at 40° C. for about 5 days. Surprisingly, powder X-ray diffraction of this sample shows that a new crystalline form is obtained which exhibits a powder pattern as shown in FIG. 3, with peak locations as indicated in Table 2 hereafter. A Raman spectrum of form B is shown in FIG. 14.

### Example B2

### Preparation of Form B from Form F

About 50 mg of DMXAA sodium salt form F, according to example F1, are stored under 75% relative humidity (corresponding to a water activity of 0.75) at 40° C. in an open powder X-ray sample holder. After three days of storage under these conditions, powder X-ray diffraction of the stored sample shows that DMXAA sodium salt form B is obtained.

### Characterization and Properties of Form B

TABLE 2
Two theta angles and d-spacings for form B

| Angle [°2θ] | d-spacings [Å] | Intensity (qualitative) |
|---|---|---|
| 8.0 | 11.1 | w |
| 8.7 | 10.2 | s |
| 9.5 | 9.3 | m |
| 12.6 | 7.0 | m |
| 13.7 | 6.5 | m |
| 15.9 | 5.57 | m |
| 16.4 | 5.41 | w |
| 17.0 | 5.21 | m |
| 17.6 | 5.04 | w |
| 19.0 | 4.67 | w |
| 19.6 | 4.53 | m |
| 20.7 | 4.29 | w |
| 20.9 | 4.25 | w |
| 21.6 | 4.12 | w |
| 21.9 | 4.05 | vw |
| 23.7 | 3.75 | m |
| 24.1 | 3.69 | w |
| 24.6 | 3.62 | s |
| 25.2 | 3.54 | vs |
| 26.3 | 3.38 | m |
| 27.5 | 3.24 | m |
| 27.9 | 3.19 | vs |
| 28.3 | 3.16 | m |

The water content of form B as determined by Karl Fischer titration or TG-FITR is 20-22%. The aqueous solubility of form B is at least 300 mg/mL at 22° C. at pH 8.3.

### 2) Preparation of Form C

### Example C1

### Preparation of Form C from Form B

About 40 mg of DMXAA sodium salt form B, according to example B1, is prepared into a PXRD sample holder and placed open into a controlled humidity measurement cell. Thereafter, the relative humidity (r. h.) is reduced from about 50% to 20% at a rate of about 5% per hour, and this relative humidity is maintained for about 45 hours. Investigation of this sample by PXRD under controlled relative humidity conditions at 20% r.h. reveals a powder X-ray diffraction pattern which is slightly different from form B. This form is here designated as form C and it shows a characteristic powder X-ray diffraction pattern as shown in FIG. 4 and peak locations as given in Table 3 hereafter. A Raman spectrum of form C is shown in FIG. 15.

### Example C2

### Preparation of Form C from Mesomorphic Form A

1.03 g of amorphous DMXAA sodium salt mesomorphic form A, according to Example A1, is dissolved in 2.6 mL of water. 1.25 mL of this solution is added to 11.0 mL of tetrahydrofuran at room temperature, which leads to immediate precipitation of a white solid product. The obtained suspension is slowly cooled to 2° C. under stirring and stirring is continued for about 18 hours before a crystalline product is obtained upon filtration and drying in air at ambient temperature. This sample is characterized by Raman spectroscopy and it shows a Raman spectrum identical to the sample according to Example C1.

### Example C3

### Preparation of Form C from Form K

270 mg of DMXAA sodium salt form K are suspended in a mixture of 3.0 mL ethyl acetate, 1.0 mL acetone and 200 μl water (i.e. 5% v/v corresponding to a water activity of about 0.50 to 0.55). The obtained suspension is stirred at ambient temperature for about 88 hours and thereafter the solid is separated by filtration. The obtained white crystalline material is dried in air at ambient temperature for about 1 hour and characterised by Raman spectroscopy, it shows a Raman spectrum identical to the sample according to Example C1.

### Example C4

### Preparation of Form C from Methanol Solvate

### (i) Preparation of Starting Material

A mixture of DMXAA and methanol (25 volumes relative to DMXAA) is treated (at 20 to 25° C.) With a methanolic solution of sodium methoxide. The mixture is warmed to 45-55° C., and stirred to dissolve. The mixture is clarified by filtration. The resulting solution is concentrated by vacuum distillation until 8 volumes of methanol (relative to DMXAA sodium salt) remain. The solution is diluted with isopropanol (15 volumes relative to DMXAA sodium salt), and the resulting mixture is cooled to 0-5° C. and stirred at 0-5° C. to crystallise. Crude DMXAA sodium salt (of undetermined form) is isolated by filtration, washed with methanol/isopropanol, and then dried under vacuum at 45° C.

### (ii) Preparation of Form C

The crude DMXAA sodium salt (see step (i) above) is stirred at ambient temperature (approximately 20° C.) for minimum 2.5 hours in a mixture of acetone (7 volumes), and water (0.5 volumes). The formed hydrate is isolated by filtration, and dried with intermittent agitation by passing a water wet nitrogen stream (the stream being obtained by passing filtered nitrogen through a reservoir of water at approximately 30° C.) through the filter cake until IPC conforms to specification (Water=18-24% w/w; acetone<4000 ppm).

### Characterization and Properties of Form C

TABLE 3
Two theta angles and d-spacings for form C

| | | Intensity |
|---|---|---|
| Angle [°2θ] | d-spacings [Å] | (qualitative) |
| 7.9 | 11.2 | m |
| 8.7 | 10.2 | vs |
| 9.9 | 9.0 | w |
| 10.2 | 8.7 | s |
| 12.8 | 6.9 | w |
| 13.1 | 6.7 | s |
| 13.6 | 6.5 | w |
| 13.8 | 6.4 | vw |
| 15.8 | 5.60 | s |
| 16.1 | 5.52 | m |
| 17.2 | 5.16 | m |
| 17.8 | 4.97 | m |
| 20.0 | 4.44 | m |
| 20.3 | 4.36 | w |
| 20.9 | 4.25 | w |
| 21.8 | 4.07 | w |
| 24.2 | 3.67 | vs |
| 25.2 | 3.54 | m |
| 25.5 | 3.49 | vs |
| 26.2 | 3.40 | s |
| 26.8 | 3.32 | s |
| 27.3 | 3.26 | vs |
| 27.8 | 3.20 | m |
| 28.9 | 3.09 | m |
| 29.4 | 3.03 | m |
| 31.9 | 2.80 | m |
| 34.0 | 2.63 | m |

The water content of form C as determined by Karl Fischer titration or TG-FITR is 15-20%.

The aqueous solubility of form C is at least 300 mg/mL at 22° C. at pH 8.3.

### Example C4

### Dynamic Vapour Sorption (DVS) Experiment

About 10 mg of a mixture of forms B and C is placed into a suitable sample pan and investigated with a dynamic vapour sorption apparatus of Surface Measurement Systems, DVS-1 (series 1000) using software version DVS win SP2, V3.01 at 25° C. The initial relative humidity is 50% and the initial mass is 100%, thereafter the humidity is increased to 95% and kept at this level for 2 hours, then the humidity is decreased to 0%, and kept at 0% during 10 hours, then raised again to 95%. Change rates are always 5% per hour.

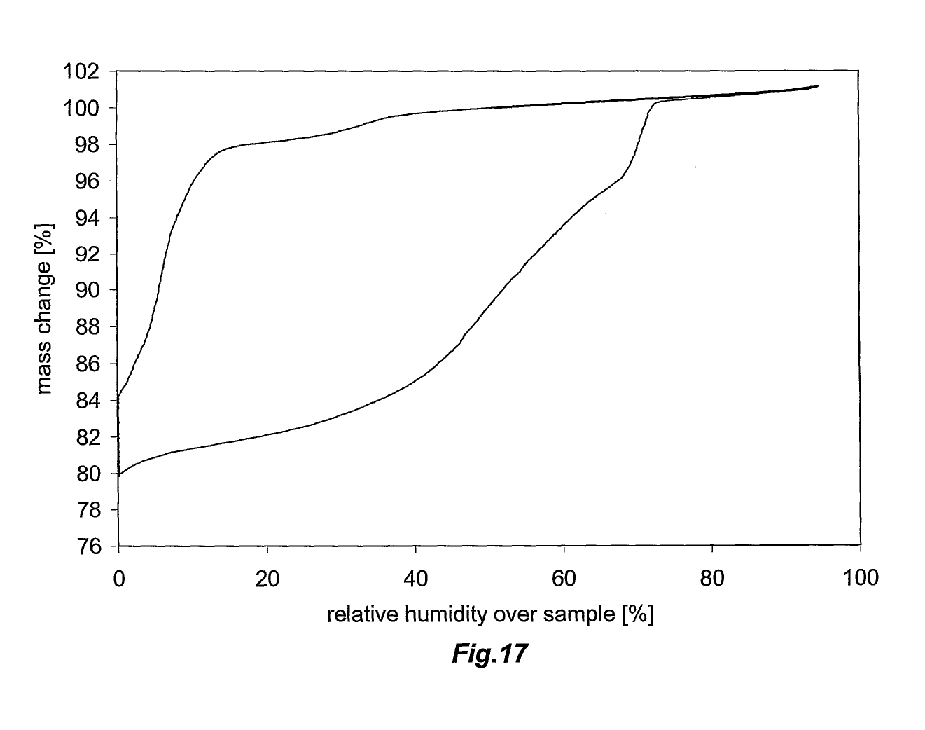

The Dynamic Vapour Sorption diagram obtained is shown in FIG. 17: it is readily visible that the initial water content is about 20%, at 95% r.h. about 1% of water is adsorbed but all the water is released upon scanning r.h. to 0%. Increasing the humidity leads to reversible water adsorption and formation of form B at 95% r.h.

Powder X-ray diffraction under controlled humidity conditions that simulate the conditions of the DVS experiment show that above about 70% r.h. form B is the dominant form. In the relative humidity range from about 20 to 60%, C is obtained and below about 5% relative humidity, form E is formed. The region from 50 to 75% relative humidity might be regarded as a transition zone where both forms B and C can exist, and the region from about 5 to 20% might be regarded as a transition zone where both forms C and E can exist.

It is clear that the interconversions these forms are entirely reversible and that there it a relatively large transition zone where forms B and C coexist.

### 3) Preparation of Form D

### Example D1

### Preparation of Form D from a Mixture of Forms B and C

649 mg of DMXAA sodium salt in form of a mixture of forms B and C are suspended in 1.0 mL of TRIS/acetate buffer (pH 8.3). This mixture is stirred at 22° C. for 50 hours before the solid is recovered by filtration. The wet cake is prepared into a 1.0 mm powder X-ray diffraction sample holder and investigated by humidity controlled powder X-ray diffraction at a relative humidity of 95%. Surprisingly, powder X-ray diffraction of this sample shows that a new crystalline form is obtained which exhibits a powder pattern as shown in FIG. 5, with peak locations as indicated in Table 4.

### Characterization and Properties of Form D

TABLE 4
Two theta angles and D-spacings for form D

| | | Intensity |
|---|---|---|
| Angle [°2θ] | d-spacings [Å] | (qualitative) |
| 7.0 | 12.6 | s |
| 7.6 | 11.7 | vs |
| 9.6 | 9.2 | vw |
| 11.0 | 8.1 | m |
| 14.0 | 6.3 | m |
| 14.9 | 5.94 | m |
| 15.7 | 5.64 | m |
| 17.5 | 5.07 | w |
| 19.1 | 4.65 | w |
| 22.7 | 3.91 | w |
| 23.0 | 3.87 | w |
| 23.2 | 3.82 | w |
| 23.9 | 3.72 | w |
| 24.9 | 3.57 | s |
| 27.5 | 3.24 | m |
| 28.0 | 3.19 | m |
| 28.7 | 3.11 | s |
| 29.2 | 3.05 | w |
| 30.0 | 2.98 | w |
| 32.1 | 2.79 | m |
| 32.5 | 2.75 | w |

The water content of form D as determined by Karl Fischer titration or TG-FITR is 23-30%.

### 4) Preparation of Form E

### Example E1

### Preparation of Form E from a Mixture of B and C

About 40 mg of DMXAA sodium salt in form of a mixture of forms B and C are prepared into a PXRD sample holder and placed open into a controlled humidity measurement cell. Thereafter the relative humidity is reduced from about 50% r.h. to 0% r.h. at a rate of 5% per hour, with about 10 hours the adsorbed water is slowly removed. Investigation of this sample by PXRD reveals that a new crystalline form is obtained. This form, which is here designated as form E exhibits a powder pattern as shown in FIG. 6, with peak locations as indicated in Table 5 hereafter.

### Characterization and Properties of Form E

TABLE 5
Two theta angles and d-spacings for form E

| | | Intensity |
|---|---|---|
| Angle [°2θ] | d-spacings [Å] | (qualitative) |
| 5.1 | 17.4 | vs |
| 6.2 | 14.1 | m |
| 7.7 | 11.5 | m |
| 8.5 | 10.4 | m |
| 8.8 | 10.1 | s |
| 9.9 | 8.9 | vs |
| 10.2 | 8.7 | vs |
| 12.4 | 7.1 | w |
| 12.6 | 7.0 | w |
| 13.7 | 6.5 | s |
| 14.1 | 6.3 | w |
| 15.4 | 5.76 | s |
| 19.8 | 4.47 | m |
| 20.4 | 4.36 | vw |
| 25.5 | 3.49 | m, broad |

From the DVS experiments reported in 2) above it is noticed that form E is an anhydrate, i.e. a water-free form.

Form E is hygroscopic. However, when kept dry in a closed container at ambient temperature form F is stable (for chemical or physical change observed after 36 months storage at ambient temperature in a tight PE packaging).

DSC analysis of form E reveals an exothermic signal near 227° C., which may show that a phase transformation under concurrent decomposition starts above 230° C. This result, as well as the fact that a phase equilibration experiment with a mixture of forms E and F in isopropanol transforms this mixture into pure form F, indicate that form E is less stable than form F.

### 5) Preparation of Form F

### Example F1

### Preparation of Form F from Mesomorphic Form A

80 mg of DMXAA sodium salt mesomorphic form A, according to Example A1, are suspended in 2.0 mL of 2-butanone in a 4.0 mL glass vial. This glass vial is placed on a laboratory shaker and the obtained suspension is shaken for about 48 hours at ambient temperature at 500 movements per minute. Then the suspension is filtered and the obtained solid it dried at 40° C. in air for 2 hours. The white crystalline material is investigated by PXRD, Raman spectroscopy, and TG-FTIR. Analysis by powder X-ray diffraction shows that a new crystalline form is obtained, which exhibits a characteristic powder X-ray diffraction pattern as shown in FIG. 7 with peak locations as indicated in Table 6 hereafter. Characterization by TG-FITR showed that the obtained solid contained about 0.8% water but essentially no residual 2-butanone.

### Example F2

### Preparation of Form F from a Mixture of B and C

368 mg of DMXAA sodium salt in form of a mixture of forms B and C are suspended in 8.0 mL of 2-butanone in a 15 mL glass vial. This suspension is first stirred at 40° C. for about 3 hours, then stirring is continued at ambient temperature for about 20 hours before the suspension is filtered and the obtained solid is dried in air at ambient temperature. 338 mg of white crystalline solid is obtained which is investigated by PXRD, Raman spectroscopy, and TG-FTIR. Analysis by powder X-ray diffraction shows the PXRD pattern of form F as depicted in FIG. 7, with peak locations as indicated in Table 6 hereafter. Characterization by TG-FITR showed that the obtained solid contained about 2% water but essentially no residual 2-butanone.

### Example F3

### Preparation of Form F from Form K

297 mg of DMXAA sodium salt form K (ethanol solvate) are suspended in 10 mL isopropanol and stirred at ambient temperature for about 18 hours before the suspension is filtered and the obtained solid is dried in air at ambient temperature. Yield: 215 mg. The off-white crystalline material is investigated by PXRD and Raman spectroscopy. Both the PXRD pattern and the obtained Raman spectrum correspond to DMXAA form F.

### Characterization and Properties of Form F

TABLE 6
Two theta angles and d-spacings for form F

| | | Intensity |
|---|---|---|
| Angle [°2θ] | d-spacings [Å] | (qualitative) |
| 7.5 | 11.7 | m |
| 9.1 | 9.7 | m |
| 9.9 | 9.0 | m |
| 10.3 | 8.5 | vw |
| 12.4 | 7.1 | w |
| 13.0 | 6.8 | w |
| 13.6 | 6.5 | vw |
| 14.7 | 6.0 | vw |
| 15.1 | 5.86 | w |
| 15.9 | 5.56 | m |
| 16.5 | 5.37 | vw |
| 17.5 | 5.07 | m |
| 18.6 | 4.76 | w |
| 19.9 | 4.45 | w |
| 22.6 | 3.93 | m |
| 23.9 | 3.72 | w |
| 24.8 | 3.58 | m |
| 25.6 | 3.48 | vs |
| 27.9 | 3.20 | w |
| 30.2 | 2.95 | m |
| 30.7 | 2.91 | m |

Form F is a solvent and water free Crystalline form (true polymorph). Form F is the most stable anhydrate form known (more stable than anhydrate form E or L).

Form F is hygroscopic. Storage of form F under 52%, relative humidity at ambient temperature for about two weeks did not lead to a change of the crystal form, but an increase of the water content to about 3% was found. At a high relative humidity, i.e. above about 70%, form F becomes unstable and converts to form B or C upon adsorption of water vapour. E.g. upon storage of form F in an open container at 75% relative humidity for about 34 hours form F is transformed into form B. The fact that upon storage at 75% relative humidity at 40° C. form F transforms into form B within a few days shows that form F is unstable against forms B and C in presence of water vapour. However, when kept dry in a closed container at ambient temperature form F is very stable.

DSC investigation of form F shows the melting point must be near 346° C., but decomposition of the sample does not allow a precise melting point and heat of fusion determination. No thermal signal that would reveal an apparent phase transition is observed below the beginning of the melting, process near 340° C.; a result that indicates that form F is of high polymorphic purity and stability.

The aqueous solubility of form F is at least 300 mg/mL at 22° C. at pH 8.3.

### 6) Preparation of Form G

### Example G1

### Preparation of Form G from Mesomorphic Form A

330 mg of DMXAA sodium salt mesomorphic form A, according to Example A1, are dissolved in 1.0 of water and the aqueous solution is added to 14.0 mL of isopropanol at room temperature. Immediately a white precipitate is formed and the obtained suspension is stirred at 40° C. for about 3.5 hours. Thereafter, the suspension is filtered and the obtained white solid is dried in air at ambient temperature for about 1.5 hours. The crystalline product is investigated by powder X-ray diffraction, Raman spectroscopy, and TG-FTIR. A powder X-ray diffraction pattern as shown in FIG. 8, with peak locations as given in Table 9 hereafter, is obtained. The obtained, sample contains some form C as phase impurity.

### Characterization and Properties of Form G

TABLE 9
Two theta angles and d-spacings for form G

| | | Intensity |
|---|---|---|
| Angle [°2θ] | d-spacings [Å] | (qualitative) |
| 5.2 | 17.0 | s |
| 7.3 | 12.0 | s |
| 8.2 | 10.7 | m |
| 10.4 | 8.5 | s |
| 11.7 | 7.6 | m |
| 14.7 | 6.0 | m |
| 15.2 | 5.82 | m |
| 15.7 | 5.65 | s |
| 16.6 | 5.35 | w |
| 16.8 | 5.26 | w |
| 18.8 | 4.70 | m |
| 20.0 | 4.45 | m |
| 20.3 | 4.37 | m |
| 21.3 | 4.17 | w |
| 22.3 | 3.98 | w |
| 23.5 | 3.78 | w |
| 24.9 | 3.58 | w |
| 26.1 | 3.41 | w |
| 26.8 | 3.32 | vs |
| 27.8 | 3.21 | s |
| 29.1 | 3.07 | m |

TG-FTIR analysis reveals that form G as obtained here contains about 10-11% of isopropanol and about 10-11% of water.

### 7) Preparation of Form H

### Example H1

### Preparation of Form H from Form C

2.00 g of DMXAA sodium salt form C, according to Example C1, is suspended in 20.0 mL of absolute ethanol in a 40 mL glass vial. The obtained suspension is stirred at 40° C. for about 48 hours before the solid is separated by filtration. The obtained white crystalline solid is dried at 60° C. for about 2 hours and thereafter investigated by powder X-ray diffraction, Raman spectroscopy, and TG-FTIR. Analysis by powder X-ray diffraction reveals an X-ray diffraction pattern as shown in FIG. 9 with peak locations as given in Table 10. Characterisation of form H by TG-FTIR shows that the obtained solid contains about 7% of water, but essentially no residual ethanol. This form is designated as form H.

### Example H2

### Preparation of Form H From DMXAA

283.2 mg of DMXAA (1.0 mmol) are, suspended in 10 mL of acetone and warmed to 40° C. At 40° C., 0.5 mL of 2.0 M NaOH aqueous solution is added to the dilute suspension of free acid. The suspension is first stirred at 40° C. for about one hour and then at ambient temperature for about 44 hours before the suspension was filtered and the obtained solid dried in air at ambient temperature. The white crystalline material is investigated by PXRD and Raman spectroscopy. Analysis by powder X-ray diffraction shows as powder X-ray diffraction pattern as shown in FIG. 9, with peak locations as indicated in Table 10 hereafter.

### Characterization and Properties of Form H

TABLE H
Two theta angles and d-spacings for form H

| | | Intensity |
|---|---|---|
| Angle [°2θ] | d-spacings [Å] | (qualitative) |
| 5.5 | 16.2 | vs |
| 6.1 | 14.5 | w |
| 6.7 | 13.2 | s |
| 8.0 | 11.1 | s |
| 9.2 | 9.6 | vs |
| 10.9 | 8.1 | s |
| 11.9 | 7.4 | w |
| 13.4 | 6.6 | s |
| 13.7 | 6.5 | vs |
| 14.6 | 6.1 | w |
| 15.2 | 5.83 | m |
| 16.3 | 5.43 | s |
| 18.2 | 4.87 | w |
| 18.4 | 4.81 | m |
| 20.1 | 4.41 | m |
| 20.7 | 4.29 | w |
| 24.5 | 3.63 | m |
| 24.8 | 3.58 | m |
| 25.8 | 3.45 | m |
| 26.9 | 3.31 | m |
| 29.5 | 3.03 | m |
| 32.3 | 2.77 | w |
| 34.6 | 2.59 | w |

TG-FTIR analysis and Karl Fischer titration reveal that form H as obtained here contains about 7-9% of water.

### 8) Preparation of Form I

### Example I1

### Preparation of Form I from Form B

660 mg of DMXAA sodium salt form B, according to example B1, are suspended in 10.0 mL of absolute ethanol in a 15 mL glass vial. The obtained suspension is stirred at 40° C. for about 18 hours, thereafter the temperature is reduced to 20° C. while stirring is continued during four hours before the solid is separated by filtration. The obtained white crystalline solid is dried in air at room temperature for about 1 hour and investigated by powder X-ray diffraction, Raman spectroscopy, and TG-FTIR. Analysis by powder X-ray diffraction reveals an X-ray diffraction pattern as shown in FIG. 10 with peak locations as given in Table 11. This form is designated as form I. Characterisation of form 1 by TG-FTIR shows that the obtained solid contains about 8.3% of water, which is removed in two distinct steps of 5.7% and 2.6% but essentially no residual ethanol.

### Characterization and Properties of Form I

TABLE 11
Two theta angles and d-spacings for form I

| | | Intensity |
|---|---|---|
| Angle [°2θ] | d-spacings [Å] | (qualitative) |
| 6.1 | 14.6 | vs |
| 8.0 | 11.1 | m |
| 9.2 | 9.7 | vs |
| 10.0 | 8.9 | w |
| 12.1 | 7.3 | s |
| 12.8 | 6.9 | m |
| 13.6 | 6.5 | w |
| 14.3 | 6.2 | m |
| 14.5 | 6.1 | w |
| 15.0 | 5.88 | m |
| 15.3 | 5.80 | m |
| 15.7 | 5.63 | w |
| 17.2 | 5.16 | m |
| 17.7 | 5.02 | w |
| 20.6 | 4.31 | m |
| 23.9 | 3.72 | m |
| 24.4 | 3.64 | s |
| 24.8 | 3.59 | s |
| 25.2 | 3.54 | m |
| 25.7 | 3.46 | m |
| 26.1 | 3.41 | m |
| 27.3 | 3.26 | s |
| 27.7 | 3.22 | s |
| 29.6 | 3.02 | m |
| 30.7 | 2.91 | m |

TG-FTIR analysis and Karl Fischer titration reveal that form I as obtained here contains about 7-9% of water.

### 9) Preparation of Form J

### Example J1

### Preparation of Form J from Mesomorphic Form A

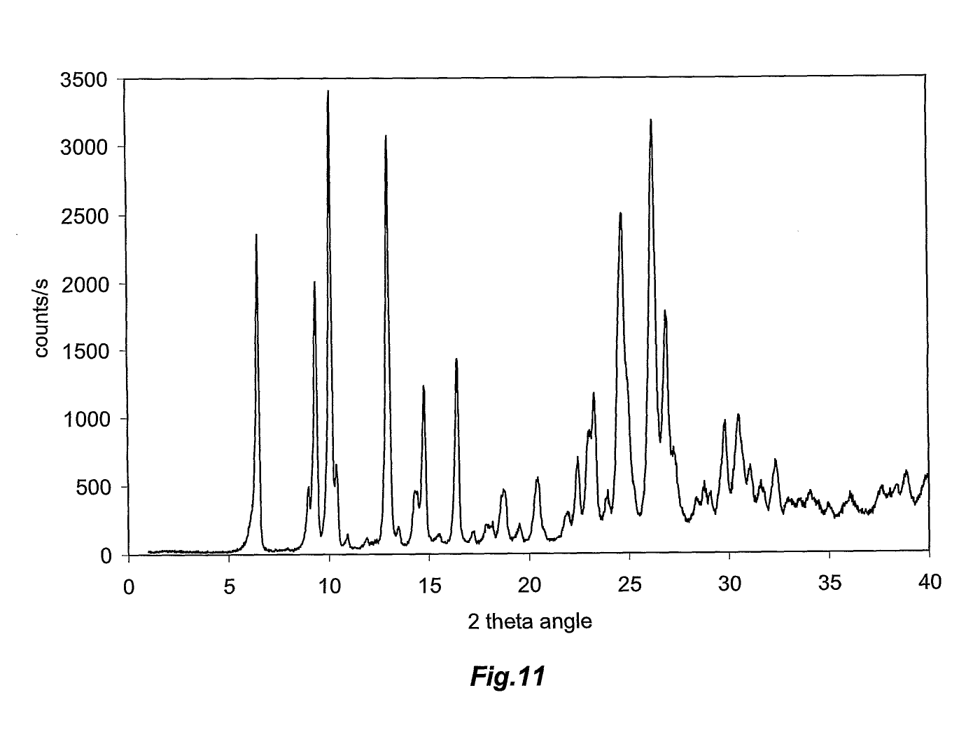

305 mg of DMXAA sodium salt mesomorphic form A, according to Example A1, are dried under nitrogen, then suspended in 10.0 mL of 1,4-dioxane in a 15 mL glass vial. The obtained suspension is stirred at 20° C. for about 20 hours before the solid is separated by filtration. The obtained white crystalline solid is dried in air at room temperature for about one hour, Investigation of the obtained product by powder X-ray diffraction shows a crystalline solid with a powder X-ray diffraction pattern essentially as shown in FIG. 11. TG-FTIR reveals a mass loss of about 27% in a distinct step near 100° C., which is attributable to release of 1,4-dioxane.

### 10) Preparation of Form K

### Example K1

### Preparation of Form K from Mesomorphic Form A

1.0 g of DMXAA sodium salt mesomorphic form A, according to Example A1, dried under nitrogen is dissolved in 20 mL of ethanol under heating, thereafter the solution is slowly cooled to 10° C. The obtained suspension is filtered, the wet cake washed with cold dry ethanol, then the obtained solid is dried, under nitrogen at room temperature for about 2 hours. Investigation of the obtained product by powder X-ray diffraction shows a crystalline solid with a powder X-ray diffraction pattern essentially as shown in FIG. 12, with peak locations as indicated in FIG. 12 hereafter. TG-FTIR reveals a mass loss of about 21.4%, occurring in two distinct steps of 6.1%, near 60° C., and 15.3% near 110° C., which are both attributable to release of ethanol.

### Characterization and Properties of Form K

TABLE 12
Two theta angles and d-spacings for form K

| | | Intensity |
|---|---|---|
| Angle [°2θ] | d-spacings [Å] | (qualitative) |
| 6.0 | 14.7 | vw |
| 7.2 | 12.3 | vs |
| 8.5 | 10.4 | s |
| 12.9 | 6.8 | m |
| 14.5 | 6.1 | m |
| 16.2 | 5.46 | w |
| 16.6 | 5.34 | m |
| 17.2 | 5.16 | w |
| 17.4 | 5.08 | m |
| 18.8 | 4.72 | w |
| 19.1 | 4.64 | w |
| 20.7 | 4.29 | w |
| 22.5 | 3.96 | w |
| 23.4 | 3.80 | w |
| 24.3 | 3.66 | w |
| 24.9 | 3.57 | m |
| 25.6 | 3.48 | w |
| 26.0 | 3.42 | m |
| 27.3 | 3.27 | m |
| 30.3 | 2.94 | m |

TG-FTIR analysis reveals that form K as obtained here contains about 21-22% of ethanol.

### 11) Preparation of Form L

### Example L1

### Preparation of Form L from Form K

260 mg of DMXAA sodium salt form K, according to Example K1, are suspended in 4.0 mL of tetrahydrofuran. This suspension is stirred at room temperature for about four days; thereafter, the suspension is filtered and the obtained solid dried at room temperature under a flow of dry nitrogen for about 2 hours. Investigation of the obtained product by powder X-ray diffraction shows a crystalline solid with a powder X-ray diffraction pattern essentially as shown in FIG. 13, with peak locations indicated in Table 13 hereafter. TG-FTIR reveals a mass loss of about 1.2% which is attributable to release of water.

### Characterization and Properties of Form L

TABLE 13
Two theta angles and d-spacings for form L

| | | Intensity |
|---|---|---|
| Angle [°2θ] | d-spacings [Å] | (qualitative) |
| 6.5 | 13.6 | w |
| 7.7 | 11.5 | m |
| 9.5 | 9.3 | m |
| 10.1 | 8.7 | w |
| 11.3 | 7.8 | vs |
| 12.0 | 7.3 | m |
| 13.0 | 6.8 | s |
| 14.8 | 6.0 | m |
| 15.1 | 5.87 | w |
| 15.4 | 5.76 | s |
| 19.4 | 4.58 | m |
| 19.9 | 4.45 | m |
| 20.2 | 4.40 | m |
| 20.9 | 4.24 | m |
| 21.1 | 4.20 | m |
| 22.5 | 3.94 | s |
| 23.2 | 3.84 | w |
| 23.5 | 3.78 | w |
| 24.7 | 3.60 | vs |
| 26.3 | 3.38 | vs |
| 29.8 | 3.00 | m |
| 30.9 | 2.89 | m |

Form L is an anhydrate, i.e. a water-free form. It is hygroscopic but stable when kept in a tight container.

Phase equilibration experiments show that form L is less stable than form F and can be used to produce that form.

It is to be understood that while the invention has been described in conjunction with the above embodiments, that the foregoing description and examples are intended to illustrate and not limit the scope of the invention. Other aspects, advantages and modifications within the scope of the invention will be apparent to those skilled in the art to which the invention pertains.

1. A crystalline DMXAA sodium salt in form of an anhydrate or a solvate.

2. A crystalline DMXAA sodium salt according to claim 1 with a physiologically acceptable solvent.

3. A crystalline DMXAA sodium salt according to claim 2 wherein the physiologically acceptable solvent is selected from the group consisting of water, ethanol and isopropanol.

4. (canceled)

5. A crystalline form of DMXAA sodium salt according to claim 3 which is a hydrate containing about 20-22% water and exhibiting a characteristic X-ray powder diffraction pattern with characteristic peaks expressed in d-values (Å): 10.2 (s), 9.3 (m), 3.54 (vs), and 3.19 (vs) hereinafter designated as form B.

6. A crystalline form of DMXAA sodium salt according to claim 5 which exhibits a characteristic X-ray powder diffraction pattern with characteristic peaks expressed in d-values (Å): 11.1 (w), 10.2 (s), 9.3 (m), 7.0 (m), 6.5 (m), 5.57 (m), 3.62 (s), 3.54 (vs), 3.38 (m), and 3.19 (vs).

7. A crystalline form of DMXAA sodium salt according to claim 6 which exhibits a characteristic X-ray powder diffraction pattern with characteristic peaks expressed in d-values (Å): 11.1 (w), 10.2 (s), 9.3 (m), 7.0 (m), 6.5 (m), 5.57 (m), 5.41 (w), 5.21 (m), 5.04 (w), 4.67 (w), 4.53 (m), 4.29 (w), 4.25 (w), 4.12 (w), 4.05 (vw), 3.75 (m), 3.69 (w), 3.62 (s), 3.54 (vs), 3.38 (m), 3.24 (m), 3.19 (vs), and 3.16 (m).

8. (canceled)

9. A crystalline form of DMXAA sodium salt according to claim 3 which is a hydrate containing about 15-20% water and exhibiting a characteristic X-ray powder diffraction pattern with characteristic peaks expressed in d-values (Å): 10.2 (vs), 8.7 (s), 5.60 (s), and 3.67 (vs) hereinafter designated as form C

10. A crystalline form of DMXAA sodium salt according to claim 9 which exhibits a characteristic X-ray powder diffraction pattern with characteristic peaks expressed in d-values (Å): 11.2 (m), 10.2 (vs), 9.0 (w), 8.7 (s), 5.60 (s), 3.67 (vs), 3.54 (m), 3.49 (vs), 3.40 (s), 3.32 (s), and 3.26 (vs).

11. A crystalline form of DMXAA sodium salt according to claim 10 which exhibits a characteristic X-ray powder diffraction pattern with characteristic peaks expressed in d-values (Å): 11.2 (m), 10.2 (vs), 9.0 (w), 8.7 (s), 6.9 (w), 6.7 (s), 6.5 (w), 6.4 (vw), 5.60 (s), 5.52 (m), 5.16 (m), 4.97 (m), 4.44 (m), 4.36 (w), 4.25 (w), 4.07 (w), 3.67 (vs), 3.54 (m), 3.49 (vs), 3.40 (s), 3.32 (s), 3.26 (vs), 3.20 (m), 3.09 (m), 3.03 (m), 2.80 (m), and 2.63 (m).

12. (canceled)

13. A crystalline form of DMXAA sodium salt according to claim 3 which is an anhydrate exhibiting a characteristic X-ray powder diffraction pattern with characteristic peaks expressed in d-values (Å): 9.7 (m), 9.0 (m), and 3.48 (vs) hereinafter designated as form F

14. A crystalline form of DMXAA sodium salt according to claim 13 which exhibits a characteristic X-ray powder diffraction pattern with characteristic peaks expressed in d-values (Å): 11.7 (m), 9.7 (m), 9.0 (m), 5.56 (m), 3.93 (m), and 3.48 (vs).

15. A crystalline form of DMXAA sodium salt according to claim 14 which exhibits a characteristic X-ray powder diffraction pattern with characteristic peaks expressed in d-values (Å): 11.7 (m), 9.7 (m), 9.0 (m), 8.5 (vw), 7.1 (w), 6.8 (w), 6.5 (vw), 6.0 (vw), 5.86 (w), 5.56 (m), 5.37 (vw), 5.07 (m), 4.76 (w), 4.45 (w), 3.93 (m), 3.72 (w), 3.58 (m), 3.48 (vs), 3.20 (w), 2.95 (m), and 2.91 (m).

16. (canceled)

17. A crystalline form of DMXAA sodium salt according to claim 3 which is a hydrate containing about 23-30% water and exhibiting a characteristic X-ray powder diffraction pattern with characteristic peaks expressed in d-values (Å): 12.6 (s), 11.7 (vs), 8.1 (m), 6.3 (m), 5.94 (m), 5.64 (m), and 3.57 (s) hereinafter designated as form D.

18. A crystalline form of DMXAA sodium salt according to claim 17 which exhibits a characteristic X-ray powder diffraction pattern with characteristic peaks expressed in d-values (Å): 12.6 (s), 11.7 (vs), 9.2 (vw), 8.1 (m), 6.3 (m), 5.94 (m), 5.64 (m), 5.07 (w), 4.65 (w), 3.91 (w), 3.87 (w), 3.82 (w), 3.72 (w), 3.57 (s), 3.24 (m), 3.19 (m), 3.11 (s), 3.05 (w), and 2.79 (m).

19. (canceled)

20. A crystalline form of DMXAA sodium salt according to claim 3 which is an anhydrate exhibiting a characteristic X-ray powder diffraction pattern with characteristic peaks expressed in d-values (Å): 17.4 (vs), 14.1 (m), 8.9 (vs), 8.7 (vs), and 5.76 (s) hereinafter designated as form E.

21. A crystalline form of DMXAA sodium salt according to claim 20 which exhibits a characteristic X-ray powder diffraction pattern with characteristic peaks expressed in d-values (Å): 17.4 (vs), 14.1 (m), 11.5 (m), 10.4 (m), 10.1 (s), 8.9 (vs), 8.7 (vs), 6.5 (s), 6.3 (w), 5.76 (s), and 3.49 (m, broad).

22. (canceled)

23. A crystalline form of DMXAA sodium salt according to claim 3 which is an ethanol solvate containing about 21-22% ethanol and exhibiting a characteristic X-ray powder diffraction pattern with characteristic peaks expressed in d-values (Å): 12.3 (vs), 10.4 (s), 6.8 (m), 6.1 (m), and 3.42 (m), hereinafter designated as form K.

24. A crystalline form of DMXAA sodium salt according to claim 23 which exhibits a characteristic X-ray powder diffraction pattern with characteristic peaks expressed in d-values (A): 14.7 (vw), 12.3 (vs), 10.4 (s), 6.8 (m), 6.1 (m), 5.46 (w), 5.34 (m), 5.16 (w), 5.08 (m), 4.72 (w), 4.64 (w), 4.29 (w), 3.96 (w), 3.80 (w), 3.66 (w), 3.57 (m), 3.48 (w), 3.42 (m), 3.27 (m), and 2.94 (m).

25. (canceled)

26. A crystalline form of DMXAA sodium salt according to claim 3 which is a hydrate containing about 7-9% water and exhibiting a characteristic X-ray powder diffraction pattern with characteristic peaks expressed in d-values (Å): 16.2 (vs), 13.2 (s), 11.1 (s), 9.6 (vs), 8.1 (s), 6.5 (vs), and), 5.43 (s), hereinafter designated as form H.

27. A crystalline form of DMXAA sodium salt according to claim 26 which exhibits a characteristic X-ray powder diffraction pattern with characteristic peaks expressed in d-values (Å): 16.2 (vs), 14.5 (w), 13.2 (s), 11.1 (s), 9.6 (vs), 8.1 (s), 7.4 (w), 6.6 (s), 6.5 (vs), 6.1 (w), 5.83 (m), 5.43 (s), 4.81 (m), 4.41 (m), 4.29 (w), 3.63 (m), 3.58 (m), 3.45 (m), 3.31 (m), 3.03 (m), 2.77 (w), and 2.59 (w).

28. (canceled)

29. A crystalline form of DMXAA sodium salt according to claim 3 which is a hydrate containing about 7-9% water and exhibiting a characteristic X-ray powder diffraction pattern with characteristic peaks expressed in d-values (Å): 14.6 (vs), 9.7 (vs), 7.3 (s), 5.88 (m), 3.64 (s), 3.59 (s), 3.26 (s), and 3.22 (s), hereinafter designated as form I.

30. A crystalline form of DMXAA sodium salt according to claim 29 which exhibits a characteristic X-ray powder diffraction pattern with characteristic peaks expressed in d-values (Å): 14.6 (vs), 11.1 (m), 9.7 (vs), 8.9 (w), 7.3 (s), 6.9 (m), 6.5 (w), 6.2 (m), 6.1 (w), 5.88 (m), 5.80 (m), 5.63 (w), 5.16 (m), 5.02 (w), 4.31 (m), 3.72 (m), 3.64 (s), 3.59 (s), 3.54 (m), 3.46 (m), 3.41 (m), 3.26 (s), 3.22 (s), 3.02 (m), and 2.91 (m).

31. (canceled)

32. A crystalline form of DMXAA sodium salt according to claim 3 which is an anhydrate exhibiting a characteristic X-ray powder diffraction pattern with characteristic peaks expressed in d-values (Å): 7.8 (vs), 6.8 (s), 5.76 (s), 3.94 (s), 3.78 (w), and 3.60 (vs), hereinafter designated as form L.

33. A crystalline form of DMXAA sodium salt according to claim 32 which exhibits a characteristic X-ray powder diffraction pattern with characteristic peaks expressed in d-values (Å): 13.6 (w), 11.5 (m), 9.3 (m), 8.7 (w), 7.8 (vs), 7.3 (m), 6.8 (s), 6.0 (m), 5.87 (w), 5.76 (s), 4.58 (m), 4.45 (m), 4.40 (m), 4.24 (m), 4.20 (m), 3.94 (s), 3.84 (w), 3.78 (w), 3.60 (vs), 3.38 (vs), 3.00 (m), and 2.89 (m).

34. (canceled)

35. A crystalline form of DMXAA sodium salt according to claim 3 which is a mixed hydrate-isopropanol solvate containing about 10-11% water and about 10-11% isopropanol and exhibiting a characteristic X-ray powder diffraction pattern with characteristic peaks expressed in d-values (Å): 17.0 (s), 12.0 (s), 8.5 (s), and 5.65 (s), hereinafter designated as form G.

36. A crystalline form of DMXAA sodium salt according to claim 35 which exhibits a characteristic X-ray powder diffraction pattern with characteristic peaks expressed in d-values (Å): 17.0 (s), 12.0 (s), 10.7 (m), 8.5 (s), 7.6 (m), 6.0 (m), 5.82 (m), 5.65 (s), 4.70 (m), 4.45 (m), 4.37 (m), 3.32 (vs), and 3.21 (s).

37. (canceled)

38. A process for preparing crystalline DMXAA sodium salt of claim 1 which is in the form of a hydrate, comprising stirring a suspension of an amorphous form or any crystalline form of DMXAA sodium salt in an organic solvent containing water as a co-solvent in a ratio that results in a suitable water activity, filtering off the obtained crystalline form and drying it under suitable conditions.

39-42. (canceled)

43. A process for preparing crystalline DMXAA sodium salt according to claim 1 which is in the form of an anhydrate, comprising suspending amorphous DMXAA sodium salt or any crystalline form of DMXAA sodium salt in an organic solvent, stirring the obtained suspension until formation of the anhydrate crystalline form is complete, filtering and drying under suitable conditions.

44. A process of preparing crystalline form F of DMXAA sodium salt anhydrate according to claim 13, comprising suspending an amorphous or any crystalline form of DMXAA sodium salt in an essentially water-free organic solvent and stirring obtained suspension until formation of form F is complete, filtering and drying in dry air at ambient temperature.

45. A process for preparing crystalline DMXAA sodium salt of claim 1 which is in the form of a solvate of a pharmaceutically acceptable organic solvent, comprising suspending an amorphous form or any crystalline form of DMXAA sodium salt in the physiologically acceptable organic solvent, stirring, filtering off the obtained crystalline form and drying it under suitable conditions.

46. A process for preparing crystalline DMXAA sodium salt of claim 1 which is in form of mixed hydrate-solvate of a physiologically acceptable organic solvent, comprising dissolving an amorphous form or any crystalline form of DMXAA sodium salt in water, adding the physiologically acceptable organic solvent, stirring, filtering off the obtained crystalline form and drying it under suitable conditions.

47-50. (canceled)

51. A pharmaceutical composition containing as active ingredient a crystalline DMXAA sodium salt in form of an anhydrate or a solvate, and a pharmaceutically acceptable carrier or diluent.

52-54. (canceled)

55. A method of preparing a pharmaceutical composition comprising, mixing a crystalline DMXAA sodium salt in an aqueous solution having a physiologically acceptable pH.

56-59. (canceled)

60. A method of treating cancer in a patient in need of such treatment, said method comprising administering to said patient an effective amount of a crystalline DMXAA sodium salt in form of an anhydrate or a solvate to the patient.

61-62. (canceled)

63. A method of treating non-small cell lung cancer in a patient in need of such treatment, said method comprising administering to said patient an effective amount of a crystalline DMXAA sodium salt in form of an anhydrate or a solvate, in combination with the sequential administration of paclitaxel and carboplatin.

64. (canceled)

65. A method of treating metastatic hormone refractory prostate cancer in a patient in need of such treatment, said method comprising administering to said patient an effective amount of a crystalline DMXAA sodium salt in form of an anhydrate or a solvate, in combination with the sequential administration of docetaxel.

66. (canceled)

67. A kit-of-parts comprising:
(a) a formulation containing a crystalline DMXAA sodium salt in form of an anhydrate or a solvate;
(b) one or more separate formulations comprising one or more further pharmaceutically active compounds selected from taxanes, platins, cyclophosphamide, vinca alkaloids, antimetabolites, topoisomerase II inhibitors, anthracyclines, tumour necrosis factor (TNF) stimulating compounds, immunomodulatory compounds, non steroidal anti-inflammatory drugs (NSAIDs), EGFR signalling pathway inhibitors and VEGF binders; and
(c) instructions for use of the formulation containing DMXAA together with said one or more separate formulations.

68-71. (canceled)

72. A pharmaceutical formulation comprising:
(a) a crystalline DMXAA sodium salt in form of an anhydrate or a solvate; and
(b) one or more further pharmaceutically active compounds selected from taxanes, platins, cyclophosphamide, vinca alkaloids, antimetabolites, topoisomerase II inhibitors, anthracyclines, tumour necrosis factor (TNF) stimulating compounds, immunomodulatory compounds, non steroidal anti-inflammatory drugs (NSAIDs), EGFR signalling pathway inhibitors and VEGF binders.

73. (canceled)
